(19) United States
(12) Patent Application Publication
(10) Pub. No.: US 2021/0393698 A1
(43) Pub. Date: Dec. 23, 2021

(54) METHODS FOR THE EXPANSION OF MESENCHYMAL STROMAL CELLS

(71) Applicant: Board of Regents, The University of Texas System, Austin, TX (US)

(72) Inventors: Elizabeth Shpall, Houston, TX (US); Katayoun Rezvani, Houston, TX (US); Mayela Mendt, Houston, TX (US)

(21) Appl. No.: 17/281,853

(22) PCT Filed: Oct. 4, 2019
(86) PCT No.: PCT/US2019/054901

§ 371 Date: Mar. 31, 2021

Related U.S. Application Data
(60) Provisional application No. 62/741,933, filed on Oct. 5, 2018.

(51) Int. Cl. A61K 35/28; A61K 35/51; C12N 5/0775

(52) U.S. Cl. CPC A61K 35/28; A61K 35/51; C12N 2501/24; C12N 2501/2301; C12N 2501/2317; C12N 5/0668

(57) ABSTRACT

Provided herein are methods for expanding populations of mesenchymal stromal cells (MSCs) comprising treating a population of MSCs derived from cord tissue with a pre-activation cytokine cocktail. Further provided herein are methods of treating immune disorders with the MSCs

This application claims the benefit of U.S. Provisional Patent Application No. 62/741,933, filed Oct. 5, 2018, the entirety of which is incorporated herein by reference.

### BACKGROUND

### 1. Field

The present invention relates generally to the fields of medicine and immunology. More particularly, it concerns expansion of mesenchymal stromal cells and uses thereof.

### 2. Description of Related Art

Over the past decade, bone marrow-derived mesenchymal stromal cells (BM-MSCs) have been used therapeutically in a variety of clinical settings including graft versus host disease, ischemic/non-ischemic cardiovascular disease, ischemic stroke and as gene delivery vehicles. Limitations with BM-MSCs include the declining number and differentiation potential of the cells with increasing donor age, the inconsistent quality of BM-MSC products and the invasiveness of the requisite BM aspiration procedure. Following a normal infant birth, the cord blood tissue (CBt) is typically discarded, thus collection of the starting material is non-invasive. CBt-MSCs can expand to higher numbers more rapidly than BM-MSCs and have similar immunosuppressive properties. Thus, there is an unmet need to develop a GMP-compliant procedure to generate large numbers of CBt-MSCs for clinical use.

### SUMMARY

In a first embodiment, the present disclosure provides methods for the expansion of CBt-derived MSCs comprising obtaining a population of MSCs from cord tissue; pre-activating the MSCs in the presence of at least three cytokines selected from the group consisting of TNFα, IFNγ, IL-1β, and IL-17; and expanding the pre-activated MSCs to obtain a population of expanded MSCs. In particular aspects, the method is GMP-compliant. In some aspects, the population of MSCs from cord tissue has been previously cryopreserved or is derived from cord tissue that is fresh or has been previously cryopreserved.

In some aspects, the obtaining comprises treating the cord tissue with an enzyme cocktail. The enzyme cocktail may comprise hyaluronidase and collagenase. In certain aspects, the collagenase is collagenase NB4/6. In additional aspects, the enzyme cocktail further comprises DNAse. The hyaluronidase may be at a concentration of 0.5 to 1.5 U/mL, such as 0.6, 0.7, 0.8, 0.9, 1.0, 1.1, 1.2, 1.3, 1.4, or 1.5 U/mL. In some aspects, the collagenase is at a concentration of 0.1 to 1 U/mL, such as 0.1, 0.2, 0.3, 0.4, 0.5, 0.6, 0.7, 0.8, 0.9, or 1 U/mL. In certain aspects, the DNAse is at a concentration of 200 to 300 U/mL, such as 200, 225, 250, 275, or 300 U/mL.

The MSCs may be cultured to at least 85% confluency, such as 86%, 87%, 88%, 89%, 90%, 91%, 92%, 93%, 94%, 95%, 96%, 97%, 98%, 99%, or 100% confluency, prior to pre-activating. In some aspects, the MSCs are cultured for 6 to 8 days, such as 6, 7, or 8 days prior to pre-activating. In certain aspects, at least 500 million, such as 600, 700, 800, 900, or 1000 million, MSCs are obtained prior to pre-activating. The pre-activating can be for 12 to 24 hours, such as 12, 13, 14, 15, 16, 17, 18, 19, 20, 21, 22, 23, or 24 hours.

In certain aspects, the MSCs are pre-activated in the presence of TNFα, IFNγ, IL-1β, and IL-17. In some aspects, TNFα, IFNγ, and/or IL-1β is at a concentration of 5 to 15 ng/mL, such as 5, 6, 7, 8, 9, 10, 11, 12, 13, 14, 15, or more ng/mL. In certain aspects, the IL-17 is present at a concentration of 20-40 ng/mL, such as 20, 25, 30, 35, 40 or more ng/mL.

In some aspects, the expansion is performed in a functionally-closed system, such as a bioreactor. For example, the bioreactor is a hollow-fiber bioreactor. In certain aspects, expansion is performed for less than 7 days, such as 6, 5, or 4 days. The MSCs may be expanded at least 50-fold, such as at least 55-, 60-, 65-, 70-, 75-, 80-fold or more. In some aspects, the MSCs have a doubling time of less than 28 hours, such as 27, 26, 25, or 24 hours. In some aspects, the method further comprises cryopreserving the expanded MSCs.

In certain aspects, the population of expanded MSCs has a higher immunosuppressive phenotype as compared to bone marrow MSCs. In some aspects, the population of expanded MSCs has a higher immunosuppressive phenotype as compared to CBt-derived MSCs expanded without cytokine pre-activation. In particular aspects, the immunosuppressive phenotype is measured by expression of anti-apoptosis factors, such as VGEF and/or TGFβ, an anti-inflammatory factor, such as TSG-6, immunomodulatory factors, and/or chemoattraction-homing factors, such as CXCR4 and CXCR3. In some aspects, the immunodulatory factors are selected from the group consisting of PD-L1, IDO, PGE2, IL-10, and TGFβ. In specific aspects, the population of expanded MSCs has increased expression of stemness markers and/or chemokine receptors as compared to BM-MSCs. Exemplary stemness markers include Nestin, Stro-1, Oct-4, Nanog and Cox-2 and chemokine receptors include VEGF, HLA-G, PGE, CXCR4, IL-10, and TGFβ. In some aspects, population of expanded MSCs have induced expression of genes associated to several immune regulatory pathways such as T cell exhaustion, granulocyte adhesion and diapedesis, antigen presentation pathway, negative regulation of immune response, positive regulation of Notch signaling, positive regulation of lymphocyte apoptotic process, agranulocyte adhesion and diapedesis, regulation of cellular response to hypoxia, TGFβ signaling, NFKβ signaling, IL-6 signaling, iNos and eNos signaling 1, positive regulation of STAT4 and PI3K signaling, and induction of T cell apoptosis. In certain aspect, the population of expanded MSCs have increased expression of genes related with diapedesis and homing including homing receptors and key adhesion molecules related to adhesion and invasion. Exemplary adhesion and invasion markers include GLG1, VCAM1, CXCR4, ICAM1, CSF3, CXCL3, CXCL8, SELPG, STAT1, IFITT3, ISG15, STAT2, MX1, OAS1, IFI6, JAK2, TAP1, IFI35, IFITM1, PSM89, IRF1, IFITM3, PTPN2, RELA, IFNAR2, HSP90AA1, JUN, ARNT, HIF1, and CUL2.

The present disclosure further provides a composition for the dissociation of cord tissue comprising collagenase, hyaluronidase, and DNase. In some aspects, the composition dissociates cord tissue for the isolation of MSCs. In certain aspects, the composition consists of collagenase, hyaluronidase, and DNase. In particular aspects, the composition does not comprise or has essentially no BSA or a trypsin inhibitor. For example, the collagenase is collagenase NB4/6. The hyaluronidase may be at a concentration of 0.5 to 1.5 U/mL, such as 0.6, 0.7, 0.8, 0.9, 1.0, 1.1, 1.2, 1.3, 1.4, or 1.5 U/mL. In some aspects, the collagenase is at a concentration of 0.1 to 1 U/mL, such as 0.1, 0.2, 0.3, 0.4, 0.5, 0.6, 0.7, 0.8, 0.9, or 1 U/mL. In certain aspects, the DNAse is at a concentration of 200 to 300 U/mL, such as 200, 225, 250, 275, or 300 U/mL. In some aspects, the CBt-derived MSCs have been previously cryopreserved.

A further embodiment provides a pharmaceutical composition comprising the expanded MSCs produced by the methods of the embodiments and a pharmaceutically acceptable carrier. Further provided herein is a composition comprising the expanded MSCs produced by the methods of the embodiments for use in the treatment of an inflammatory disorder. In some aspects, the inflammatory disease is graft versus host disease (GVHD), an autoimmune disease, acute ischemic stroke, myocardial damage, acute respiratory distress syndrome (ARDS), or inflammatory bowel disease. In particular aspects, the MSCs are allogeneic. The MSCs may be administered systemically or locally.

In another embodiment, there is provided a method of treating an inflammatory disease in a subject comprising administering to said subject a therapeutically effective amount of the CBt-derived MSCs, such as the CBt-MSCs produced according to the present embodiments. In particular aspects, the subject is human. In some aspects, the CBt-derived MSCs have been previously cryopreserved.

In some aspects, the inflammatory disease is GVHD, an autoimmune disease, acute ischemic stroke, myocardial damage, ARDS, or inflammatory bowel disease. In particular aspects, the MSCs are allogeneic. The MSCs may be administered systemically or locally. For example, the MSCs are administered via the rectal, nasal, buccal, vaginal, subcutaneous, intracutaneous, intravenous, intraperitoneal, intramuscular, intraarticular, intrasynovial, intrasternal, intrathecal, intralesional, or intracranial route, or via an implanted reservoir. In additional aspects, the MSCs are administered in conjunction with at least one additional therapeutic agent. In some aspects, the at least one additional therapeutic agent is a therapeutically effective amount of an immunomodulatory or an immunosuppressive agent. In particular aspects, the immunosuppressive agent is a calcineurin inhibitor, an mTOR inhibitor, an antibody, a chemotherapeutic agent irradiation, a chemokine, an interleukin or an inhibitor of a chemokine or an interleukin.

A further embodiment provides the use of a therapeutically effective amount of CBt-derived MSCs, such as the CBt-MSCs produced according to the present embodiments for the treatment of an inflammatory disorder. In some aspects, the CBt-derived MSCs have been previously cryopreserved. In some aspects, the inflammatory disease is GVHD, an autoimmune disease, acute ischemic stroke, myocardial damage, ARDS, or inflammatory bowel disease. In particular aspects, the MSCs are allogeneic. The MSCs may be administered systemically or locally. For example, the MSCs are administered via the rectal, nasal, buccal, vaginal, subcutaneous, intracutaneous, intravenous, intraperitoneal, intramuscular, intraarticular, intrasynovial, intrasternal, intrathecal, intralesional, or intracranial route, or via an implanted reservoir. In additional aspects, the MSCs are administered in conjunction with at least one additional therapeutic agent. In some aspects, the at least one additional therapeutic agent is a therapeutically effective amount of an immunomodulatory or an immunosuppressive agent. In particular aspects, the immunosuppressive agent is a calcineurin inhibitor, an mTOR inhibitor, an antibody, a chemotherapeutic agent irradiation, a chemokine, an interleukin or an inhibitor of a chemokine or an interleukin.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present disclosure. The present disclosure may be better understood by reference to one or more of these drawings in combination with the detailed description of specific embodiments presented herein.

FIG. 1: Schematic depicting GMP-compliant protocol for the isolation and expansion of MSCs from umbilical cord tissue.

FIG. 2: Schematic depicting GMP-compliant protocol for the expansion and generation of pre-activated MSCs from cord tissue using a bioreactor.

FIG. 3: Data of large-scale expansion of MSCs from bone marrow vs cord tissue in a Terumo Bioreactor.

FIG. 4: Flow cytometry analysis showing MSCs from cord tissue express higher levels of the stemness markers than MSCs from bone marrow.

FIGS. 5A-5B: Pre-activated CBt-MSCs exhibit a higher suppressive effect on T cell proliferation and activation than untreated CBt-MSCs. (FIG. 5A) Suppression of CD4⁺ T cell proliferation and (FIG. 5B) activation mediated by untreated MSCs vs pre-activated MSCs.

FIGS. 6A-6C: Pre-activation of CBt-MSCs enhances their immunosuppressive therapeutic potential. (FIG. 6A) Pre-activated CBt-MSCs show a higher immunosuppressive phenotype than pre-activated bone marrow MSCs. (FIG. 6B) Pre-activation of MSCs increases the secretion of immunoregulatory molecule TGS-6. (FIG. 6C) Pre-activation of CBt-MSCs induces the expression of immunomodulatory molecules on their surface and maximizes their therapeutic effect.

FIG. 7: Pre-activation of CBt-MSCs with the present cocktail of cytokines (TNF, IFN, ILL and IL-17) had a greater therapeutic effect than a commercial preparation.

FIGS. 8A-8C: Fresh CBt-derived MSCs increased survival in a xenogeneic graft versus host disease (GVHD) mouse model. (FIG. 8A) A significant increase in the overall survival of the mice who received either fresh BM or CBT-derived MSCs compared with the GVHD controls is shown. (FIG. 8B) Histopathological samples which demonstrate a slight reduction in GVHD signs of liver, spleen and colon of the mice treated (with either BM- or CBT-MSCs) compared with the GVHD control. (FIG. 8C) A short-term biodistribution experiment using DiR-immunofluorescence labeled MSCs infused via tail vein into the mice. CBt-MSCs showed a significant improvement in persistence compared to BM-MSCs over the course of 72 hours.

FIGS. 9A-9B: Activation of CBt-MSCs revealed a unique profile with higher immunosuppressive properties. (FIG. 9A) Heat map of genes differentially expressed between resting and activated MSCs, showed the upregulation of 816 genes and down regulation of 383 genes in activated CBt-MSCs compared with the resting CBt-MSCS. (FIG. 9B) Ingenuity Pathway Analysis (IPA) of the genes evaluated on resting and activated CBt-MSCs revealed that activation of cells induced the expression of genes associated to several immune regulatory pathways such as T cell exhaustion, negative regulation of immune response, IL-6 signaling, and induction of T cell apoptosis.

FIGS. 10A-10H: Activation enhanced CBt-MSC homing and biodistribution in a GVHD xenograft mice model. (FIG. 10A) Heat map of IPA analysis performed on the RNA extracted from activated vs resting CBt-MSCs revealed the activation of several genes related with diapedesis and homing including homing receptors and key adhesion molecules related to adhesion and invasion on the activated CBt MSCs. (FIG. 10B) Heat map of homing receptors, adhesion molecules, and invasion proteins (metalloproteinases) on activated CBT MSCs vs. resting MSCs, which were evaluated by flow cytometry. (FIGS. 10C-10D) After 72 hours fluorescence analysis, it was observed that activated CBT-MSCs persisted in the mice longer than control CBt-MSC for up to three days. (FIG. 10E) Mouse tissues were harvested at 3 h, 48 h, and 72 h post-injection and the average radiant efficiency was calculated by tissue. A trend toward higher fluorescence level was shown for the activated CBt-MSCs group compared to control MSC group, as shown in (FIG. 10F) for the lung, (FIG. 10G) for the liver, and (FIG. 10H) for the spleen.

FIGS. 11A-11C: Cryopreserved activated CBt-MSCs demonstrated a similar viability, phenotype, and efficacy controlling T cell activation compared to fresh activated CBt-MSCs. (FIG. 11A) Representative FACS plot of the viability of CBt-MSCs determined by flow cytometry using annexin V and propidium iodide assay. (FIG. 11B) Representative histogram of a T cell proliferation CFSE assay, demonstrating the total suppression of T cell activated with CD3/CD28 beads in all the different ratios. (FIG. 11C) Representative FACS plot of activated MSCs phenotype with either fresh or frozen/thawed cells showing the persistence of the expression of immunosuppressive factors.

FIGS. 12A-12G: Cryopreserved activated CBt-MSCs increased the overall survival and reduced GVHD toxicities. FIGS. 12A-12C summarize the in vivo experiment with groups of mice (n=8 mice per group). (FIG. 12A) Survival curves for the untreated (GVHD controls), recipients of unactivated CBt-MSCs, and recipients activated CBt-MSCs. The data demonstrates a survival benefit for the recipients of the activated CBT-MSCs compared to unactivated MSCs or controls. (FIG. 12B) Percent weight variations demonstrating less weight loss for the recipients of activated CBt-MSCs versus the other two groups. (FIG. 12C) Average GVHD scores, again showing less GVHD for the activated CBT-MSC group. (FIG. 12D) Portal liver inflammation compared among the three groups of mice at the end time point. (FIG. 12E) Comparison of hematological tests from mice that were untreated (control), treated with resting CBt-MSCs versus the activated (activated) MSCs. The blood tests include WBC count, MCV, MCHC, Hematocrit, Hemoglobin, Platelet count, RBC count, MCH, RDW, Albumin, Alkaline phosphatase, potassium, LDH, AST, Glucose, ALT, Phosphorus, total protein. Results show an improvement in the platelet count, glucose, WBC count, and liver function (ALT, AST) in the group treated with activated MSCs compared with the control or unactivated MSC groups. (FIG. 12F) Results of the cytokine levels in the blood of the mice, revealing that both the activated and unactivated CBt-MSCs reduced the presence of inflammatory cytokines compared to the control mice. (FIG. 12G) Percentage of human CD45 in the blood of the mice on Day 24. The left panel shows a representative FACS plot from each group, while the right panel shows a bar plot with statistical comparison compared to control group (untreated), with ** p-value<0.01, **** p-value<0.0001 demonstrating fewer human CD45⁺ cells in the blood of both MSC recipient groups with the activated MSCs showing fewer than unactivated MSC recipients.

### DESCRIPTION OF ILLUSTRATIVE EMBODIMENTS

Bone marrow-derived MSCs have been used for many years to treat refractory graft versus host disease (GVHD) and more recently in the settings of regenerative medicine including ischemic stroke, cardiovascular disease, inflammatory bowel disease, and acute respiratory distress syndrome. Cord blood from the placental vein has been evaluated extensively and is a suboptimal source of MSCs with very inconsistent and scant MSC generation compared to bone marrow. Thus, certain embodiments of the present disclosure provide methods for the expansion of MSCs derived from cord tissue. The present methods provide a robust good manufacturing practice (GMP)-compliant method to generate large doses of CBt-derived MSCs in a bioreactor.

In particular, the present methods for expanding MSCs may comprise a pre-activation step which results in the generation of CBt-derived MSCs which are significantly more suppressive than those generated without pre-activation. The pre-activation step may comprise culturing the MSCs in the presence of cytokines, such as TNFα, IFNγ, IL-1β, and IL-17. Thus, the present methods can generate CBt-derived MSCs in larger doses than BM-derived MSCs in shorter periods of time using the novel GMP-compliant system. The CBt-MSCs can thus be generated more cheaply and efficiently than BM-derived MSCs.

In the present studies, the pre-activated and expanded MSCs were found to express more markers of “stemness” than BM-derived MSCs. The increased expression of the stemness markers may allow for the ability of the pre-activated and expanded MSCs to provide more specific regeneration of vital organs including the brain, heart, gastrointestinal tract and lung. The present pre-activated MSCs also express higher levels of immunosuppressive factors and chemokine receptors that can enhance their ability to home to sites of inflammation including the gastrointestinal tract and skin in GVHD as well as to the brain and heart in regenerative medicine settings where acute inflammation is operative including VEGF, HLA-G, PGE, CXCR4, IL-10, and TGFβ. It was also found that the activated MSCs produced by the present methods had induced expression of genes associated to several immune regulatory pathways such as T cell exhaustion, granulocyte adhesion and diapedesis, antigen presentation pathway, negative regulation of immune response, positive regulation of Notch signaling, positive regulation of lymphocyte apoptotic process, agranulocyte adhesion and diapedesis, regulation of cellular response to hypoxia, TGFβ signaling, NFKβ signaling, IL-6 signaling, iNos and eNos signaling 1, positive regulation of STAT4 and PI3K signaling, and induction of T cell apoptosis. The activated MSCs also showed activation of several genes related with diapedesis and homing including homing receptors and key adhesion molecules related to adhesion and invasion on the activated CBt MSCs, such as GLG1, VCAM1, CXCR4, ICAM1, CSF3, CXCL3, CXCL8, SELPG, STAT1, IFITT3, ISG15, STAT2, MX1, OAS1, IFI6, JAK2, TAP1, IFI35, IFITM1, PSM89, IRF1, IFITM3, PTPN2, RELA, IFNAR2, HSP90AA1, JUN, ARNT, HIF1, and CUL2.

The present system can be used to generate a large number of clinical-grade CBt-derived MSCs in a GMP-compliant, functionally-closed system for infusion into patients as regenerative medicine. Accordingly, further provided herein are methods for the use of the highly immunosuppressive MSCs provided herein, such as for the treatment of inflammatory states, such as GVHD and autoimmune disease, as well as in regenerative medicine settings including acute ischemic stroke, myocardial damage, acute respiratory distress syndrome (ARDS), and inflammatory bowel disease.

### I. DEFINITIONS

As used herein, “essentially free,” in terms of a specified component, is used herein to mean that none of the specified component has been purposefully formulated into a composition and/or is present only as a contaminant or in trace amounts. The total amount of the specified component resulting from any unintended contamination of a composition is therefore well below 0.05%, preferably below 0.01%. Most preferred is a composition in which no amount of the specified component can be detected with standard analytical methods.

As used herein the specification, “a” or “an” may mean one or more. As used herein in the claim(s), when used in conjunction with the word “comprising,” the words “a” or “an” may mean one or more than one.

The use of the term “or” in the claims is used to mean “and/or” unless explicitly indicated to refer to alternatives only or the alternatives are mutually exclusive, although the disclosure supports a definition that refers to only alternatives and “and/or.” As used herein “another” may mean at least a second or more. The term “about” refers to the stated value plus or minus 5%.

The term “mesenchymal stem cell,” “mesenchymal stromal cell,” or “MSC”, as used herein, refers to a multipotent somatic stem cell derived from mesoderm, having self-regenerating and differentiating capacity to produce progeny cells with a large phenotypic variety, including connective tissues, stroma of bone marrow, adipocytes, dermis and muscle, among others. MSCs generally have a cell marker expression profile characterized in that they are negative for the markers CD19, CD45, CD14 and HLA-DR, and positive for the markers CD105, CD106, CD90 and CD73. MSCs may be isolated from any type of tissue. Generally, MSCs will be isolated from bone marrow, adipose tissue, umbilical cord, or peripheral blood. In a particular embodiment, the MSC are bone marrow-derived stem cells.

The term “functionally closed” refers to a system sealed to ensure fluid sterility either by hermetically sealing the entire system or by providing sterile barrier filters at all connections to the collection system

The term “bioreactor” refers to a large-scale cell culture system that provides nutrients to cells and removes metabolites, as well as furnishes a physio-chemical environment conducive to cell growth, in a closed sterile system. In particular aspects, the biological and/or biochemical processes develop under monitored and controlled environmental and operating conditions, for example, pH, temperature, pressure, nutrient supply and waste removal. According to the present disclosure, the basic class of bioreactors suitable for use with the present methods includes hollow fiber bioreactors.

The term “hollow fiber” is intended to include hollow structures (of any shape) containing pores of defined size, shape and density for use in delivering nutrients (in solution) to cells contained within a bioreactor and for removal of waste materials (in solution) from cells contained within a bioreactor. For purposes of the present disclosure, hollow fibers may be constructed of a resorbable or nonresorbable material. Fibers include, but are not limited to, tubular structures.

An “immune disorder,” “immune-related disorder,” or “immune-mediated disorder” refers to a disorder in which the immune response plays a key role in the development or progression of the disease Immune-mediated disorders include autoimmune disorders, allograft rejection, graft versus host disease and inflammatory and allergic conditions.

An “autoimmune disease” refers to a disease in which the immune system produces an immune response (for example, a B-cell or a T-cell response) against an antigen that is part of the normal host (that is, an autoantigen), with consequent injury to tissues. An autoantigen may be derived from a host cell, or may be derived from a commensal organism such as the micro-organisms (known as commensal organisms) that normally colonize mucosal surfaces.

The term “Graft-Versus-Host Disease (GVHD)” refers to a common and serious complication of bone marrow transplantation wherein there is a reaction of donated immunologically competent lymphocytes against a transplant recipient's own tissue. GVHD is a possible complication of any transplant that uses or contains stem cells from either a related or an unrelated donor. In some embodiments, the GVHD is chronic GVHD (cGVHD) and in some embodiments the GVHD is acute GVHD (aGVHD).

A “parameter of an immune response” is any particular measurable aspect of an immune response, including, but not limited to, cytokine secretion (IL-6, IL-10, IFN-γ, etc.), chemokine secretion, altered migration or cell accumulation, immunoglobulin production, dendritic cell maturation, regulatory activity, number of regulatory B cells and proliferation of any cell of the immune system. Another parameter of an immune response is structural damage or functional deterioration of any organ resulting from immunological attack. One of skill in the art can readily determine an increase in any one of these parameters, using known laboratory assays. In one specific non-limiting example, to assess cell proliferation, incorporation of ³H-thymidine can be assessed. A “substantial” increase in a parameter of the immune response is a significant increase in this parameter as compared to a control. Specific, non-limiting examples of a substantial increase are at least about a 50% increase, at least about a 75% increase, at least about a 90% increase, at least about a 100% increase, at least about a 200% increase, at least about a 300% increase, and at least about a 500% increase. Similarly, an inhibition or decrease in a parameter of the immune response is a significant decrease in this parameter as compared to a control. Specific, non-limiting examples of a substantial decrease are at least about a 50% decrease, at least about a 75% decrease, at least about a 90% decrease, at least about a 100% decrease, at least about a 200% decrease, at least about a 300% decrease, and at least about a 500% decrease. A statistical test, such as a non-parametric ANOVA, or a T-test, can be used to compare differences in the magnitude of the response induced by one agent as compared to the percent of samples that respond using a second agent. In some examples, p≤0.05 is significant, and indicates that the chance that an increase or decrease in any observed parameter is due to random variation is less than 5%. One of skill in the art can readily identify other statistical assays of use.

“Treating” or treatment of a disease or condition refers to executing a protocol, which may include administering one or more drugs to a patient, in an effort to alleviate signs or symptoms of the disease. Desirable effects of treatment include decreasing the rate of disease progression, ameliorating or palliating the disease state, and remission or improved prognosis. Alleviation can occur prior to signs or symptoms of the disease or condition appearing, as well as after their appearance. Thus, “treating” or “treatment” may include “preventing” or “prevention” of disease or undesirable condition. In addition, “treating” or “treatment” does not require complete alleviation of signs or symptoms, does not require a cure, and specifically includes protocols that have only a marginal effect on the patient.

The term “therapeutic benefit” or “therapeutically effective” as used throughout this application refers to anything that promotes or enhances the well-being of the subject with respect to the medical treatment of this condition. This includes, but is not limited to, a reduction in the frequency or severity of the signs or symptoms of a disease. For example, treatment of cancer may involve, for example, a reduction in the size of a tumor, a reduction in the invasiveness of a tumor, reduction in the growth rate of the cancer, or prevention of metastasis. Treatment of cancer may also refer to prolonging survival of a subject with cancer.

“Subject” and “patient” refer to either a human or non-human, such as primates, mammals, and vertebrates. In particular embodiments, the subject is a human

As generally used herein “pharmaceutically acceptable” refers to those compounds, materials, compositions, and/or dosage forms which are, within the scope of sound medical judgment, suitable for use in contact with the tissues, organs, and/or bodily fluids of human beings and animals without excessive toxicity, irritation, allergic response, or other problems or complications commensurate with a reasonable benefit/risk ratio.

“Pharmaceutically acceptable salts” means salts of compounds disclosed herein which are pharmaceutically acceptable, as defined above, and which possess the desired pharmacological activity. Such salts include acid addition salts formed with inorganic acids such as hydrochloric acid, hydrobromic acid, sulfuric acid, nitric acid, phosphoric acid, and the like; or with organic acids such as 1,2-ethanedisulfonic acid, 2-hydroxyethanesulfonic acid, 2-naphthalenesulfonic acid, 3-phenylpropionic acid, 4,4′-methylenebis(3-hydroxy-2-ene-1-carboxylic acid), 4-methylbicyclo[2.2.2]oct-2-ene-1-carboxylic acid, acetic acid, aliphatic mono- and dicarboxylic acids, aliphatic sulfuric acids, aromatic sulfuric acids, benzenesulfonic acid, benzoic acid, camphorsulfonic acid, carbonic acid, cinnamic acid, citric acid, cyclopentanepropionic acid, ethanesulfonic acid, fumaric acid, glucoheptonic acid, gluconic acid, glutamic acid, glycolic acid, heptanoic acid, hexanoic acid, hydroxynaphthoic acid, lactic acid, laurylsulfuric acid, maleic acid, malic acid, malonic acid, mandelic acid, methanesulfonic acid, muconic acid, o-(4-hydroxybenzoyl)benzoic acid, oxalic acid, p-chlorobenzenesulfonic acid, phenyl-substituted alkanoic acids, propionic acid, p-toluenesulfonic acid, pyruvic acid, salicylic acid, stearic acid, succinic acid, tartaric acid, tertiarybutylacetic acid, trimethylacetic acid, and the like. Pharmaceutically acceptable salts also include base addition salts which may be formed when acidic protons present are capable of reacting with inorganic or organic bases. Acceptable inorganic bases include sodium hydroxide, sodium carbonate, potassium hydroxide, aluminum hydroxide and calcium hydroxide. Acceptable organic bases include ethanolamine, diethanolamine, triethanolamine, tromethamine, N-methylglucamine and the like. It should be recognized that the particular anion or cation forming a part of any salt of this invention is not critical, so long as the salt, as a whole, is pharmacologically acceptable. Additional examples of pharmaceutically acceptable salts and their methods of preparation and use are presented in Handbook of Pharmaceutical Salts: Properties, and Use (P. H. Stahl & C. G. Wermuth eds., Verlag Helvetica Chimica Acta, 2002).

A “pharmaceutically acceptable carrier,” “drug carrier,” or simply “carrier” is a pharmaceutically acceptable substance formulated along with the active ingredient medication that is involved in carrying, delivering and/or transporting a chemical agent. Drug carriers may be used to improve the delivery and the effectiveness of drugs, including for example, controlled-release technology to modulate drug bioavailability, decrease drug metabolism, and/or reduce drug toxicity. Some drug carriers may increase the effectiveness of drug delivery to the specific target sites. Examples of carriers include: liposomes, microspheres (e.g., made of poly(lactic-co-glycolic) acid), albumin microspheres, synthetic polymers, nanofibers, protein-DNA complexes, protein conjugates, erythrocytes, virosomes, and dendrimers.

The term “culturing” refers to the in vitro maintenance, differentiation, and/or propagation of cells in suitable media. By “enriched” is meant a composition comprising cells present in a greater percentage of total cells than is found in the tissues where they are present in an organism

An “isolated” biological component (such as a portion of hematological material, such as blood components) refers to a component that has been substantially separated or purified away from other biological components of the organism in which the component naturally occurs. An isolated cell is one which has been substantially separated or purified away from other biological components of the organism in which the cell naturally occurs.

As used herein, the term “substantially” is used to represent a composition comprising at least 80% of the desired component, more preferably 90% of the desired component, or most preferably 95% of the desired component. In some embodiments, the composition comprises at least 80%, 82%, 84%, 86%, 88%, 90%, 91%, 92%, 93%, 94%, 95%, 96%, 97%, or 98% of the desired component.

### II. MESENCHYMAL STROMAL CELLS

The present disclosure concerns the expansion of MSCs. The MSCs used in culture can include cells derived from any stem cell source, such as umbilical cord, umbilical cord blood, placenta, embryonic stem cells, adipose tissue, bone marrow, or other tissue-specific mesenchyme. These samples may be fresh, frozen, or refrigerated. In particular aspects, the MSCs are derived from cord tissue and methods for the expansion of these CBt-derived MSCs. In particular aspects, the MSCs are human MSCs, which may autologous or allogeneic.

A. Isolation of MSCs from Cord Tissue

In one embodiment, MSCs are isolated in the presence of one or more enzyme activities. A broad range of digestive enzymes for use in cell isolation from tissue are known in the art, including enzymes ranging from those considered weakly digestive (e.g. deoxyribonucleases and the neutral protease, dispase) to strongly digestive (e.g. papain and trypsin). Presently preferred are mucolytic enzyme activities, metalloproteases, neutral proteases, serine proteases (such as trypsin, chymotrypsin, or elastase), and deoxyribonucleases. More preferred are enzyme activities selected from metalloproteases, neutral proteases and mucolytic activities. Cells can be isolated in the presence of one or more activities of collagenase, hyaluronidase and dispase.

Cord tissue may be obtained from a mammal, such as a human. In particular, the cord tissue is obtained from a full-term neonate following elective cesarean section. The cord tissue can be transported in plasmalyte, such as with penicillin/streptomycin. The cord tissue can then be cut into fractions and incubated, such as for 30-90 minutes, particularly 70, 71, 72, 73, 74, 75, 76, 77, 78, 79, or 80 minutes, at 30-40° C., particularly 37° C.

The cord tissue may be dissociated in an enzyme cocktail provided herein comprising collagenase, hyaluronidase, and/or DNAse. In particular, the collagenase is collagenase-NB4/6 (Serva). The cord tissue can then be dissociated in a dissociator, such as the GentleMACS Octo Dissociator (Miltenyi). The cell suspension is then filtered, washed and resuspended in media, such as alpha-MEM media containing 10% Platelet lysate, L-glutamine, heparin (complete media) with pen-strep and seeded into T175 flasks, then cultured until the MSCs are about 80% confluent. The cells are then harvested and expanded to about 80% confluence using complete media without antibiotics. The culturing may be for about 6 to 8 days, particularly about 7 days.

The cell culture surfaces for MSC culture include but are not limited to standard tissue culture vessels and two-dimensional surfaces, including sheets, slides, culture dishes, culture flasks, bags, culture bottles, or multiwell dishes.

Providing the growth conditions allows for a wide range of options as to culture medium, supplements, atmospheric conditions, and relative humidity for the cells. A preferred temperature is 37° C.; however, the temperature may range from about 35° C. to 39° C. depending on the other culture conditions and desired use of the cells or culture.

The skilled artisan will appreciate that the Growth Medium can be variously supplemented and altered in any of the ways known in the art, and may be optimized for particular reasons. In addition, the cells are able to grow in many other culture media, including chemically defined media in the absence of added serum. Several such media are exemplified below. In addition to routine culturing and maintenance of the cells, many other media are known in the art for affecting differentiation of such potent cells into specific types of cells or progenitors of specific cells. The skilled artisan will appreciate that these media are useful for many purposes, and are included within the scope of the invention, but they are not necessarily preferred for routine culturing and expansion.

In addition to the flexibility of the cells with respect to culturing medium, the cells can grow under a variety of environmental conditions. In particular, the cells can grow under a wide range of atmospheric conditions. Presently preferred are atmospheres which range from about 5% O₂ to about 20% or more O₂. The cells grow and expand well in Growth Medium under these conditions, typically in the presence of about 5% CO_{2,} and the balance of the atmosphere as nitrogen. The skilled artisan will appreciate that the cells may tolerate broader ranges of conditions in different media, and that optimization for specific purposes may be appropriate.

Cryopreservation of cells prior to culture or cryopreservation of expanded cells disclosed herein may be carried out according to known methods. For example, cells may be suspended in a “freezing medium” such as, for example, culture medium further comprising 10% dimethylsulfoxide (DMSO), with or without 5-10% glycerol, at a density, for example, of about 1-2×10⁶ cells/ml. The cells may be dispensed into glass or plastic vials, which are then sealed and transferred to a freezing chamber of a programmable or passive freezer. The optimal rate of freezing may be determined empirically. For example, a freezing program that gives a change in temperature of about −1° C./min through the heat of fusion may be used. Once vials containing the cells have reached −80° C., they may be transferred to a liquid nitrogen storage area.

In some embodiments, freshly isolated cells from any stem cell source may be cryopreserved to constitute a bank of cells, portions of which may be withdrawn by thawing and then used to produce the expanded cells of the invention as needed. Thawing may be carried out rapidly, for example, by transferring a vial from liquid nitrogen to a 37° C. water bath. The thawed contents of the vial may be immediately transferred under sterile conditions to a culture vessel containing an appropriate medium such as nutritive medium. Once in culture, the cells may be examined daily, for example, with an inverted microscope to detect cell proliferation, and subcultured as soon as they reach an appropriate density.

Cells may be withdrawn from a cell bank as needed, and used for the production of new stem cells or tissue either in vitro, for example, as a three-dimensional scaffold culture, or in vivo, for example, by direct administration of cells to the site where tissue reconstitution or repair is needed. As described herein, the expanded MSCs of the present disclosure may be used to reconstitute or repair tissue in a subject where the cells were originally isolated from that subject's own tissue (i.e., autologous cells). Alternatively, the expanded MSCs disclosed herein may be used as ubiquitous donor cells to reconstitute or repair tissue in any subject (i.e., heterologous cells).

B. MSC Pre-activation

The MSCs isolated from cord tissue may then be cultured in the presence of cytokines for pre-activation. The cytokines may be TNFα, IFNγ, IL-1β, and/or IL-17 and in particular are TNFα, IFNγ, IL-1β, and IL-17. The pre-activation step may be for about 12 to 24 hours, such as 13, 14, 15, 16, 17, 18, or 19 hours, particularly 16 hours. The TNFα, IFNγ, and/or IL-1β may be at a concentration of 5 to 15 ng/mL, such as 6, 7, 8, 9, 10, 11, 12, 13, or 14 ng/mL, particularly about 10 ng/mL. The IL-17 may be present at a concentration of 20-40 ng/mL, such as 25, 30, or 35 ng/mL, particular about 30 ng/mL.

C. MSC Expansion in Bioreactors

The MSCs may then be expanded in a functionally closed system, such as a bioreactor. Expansion may be performed in a Quantum Bioreactor (Terumo), such as for 4-10 days, particularly for 5-6 days.

Bioreactors can be grouped according to general categories including: static bioreactors, stirred flask bioreactors, rotating wall vessel bioreactors, hollow fiber bioreactors and direct perfusion bioreactors. Within the bioreactors, cells can be free, or immobilized, seeded on porous 3-dimensional scaffolds (hydrogel).

Hollow fiber bioreactors can be used to enhance the mass transfer during culture. A Hollow fiber bioreactor is a 3D cell culturing system based on hollow fibers, which are small, semi-permeable capillary membranes arranged in parallel array with a typical molecular weight cut-off (MWCO) range of 10-30 kDa. These hollow fiber membranes are often bundled and housed within tubular polycarbonate shells to create hollow fiber bioreactor cartridges. Within the cartridges, which are also fitted with inlet and outlet ports, are two compartments: the intracapillary (IC) space within the hollow fibers, and the extracapillary (EC) space surrounding the hollow fibers.

Thus, for the present disclosure, the bioreactor may be a hollow fiber bioreactor. Hollow fiber bioreactors may have the cells embedded within the lumen of the fibers, with the medium perfusing the extra-lumenal space or, alternatively, may provide gas and medium perfusion through the hollow fibers, with the cells growing within the extralumenal space. Hollow fiber bioreactors suitable for the present disclosure are known in the art and may include, but are not limited to, the Caridian (Terumo) BCT Quantum Cell Expansion System.

The hollow fibers should be suitable for the delivery of nutrients and removal of waste in the bioreactor. The hollow fibers may be any shape, for example, they may be round and tubular or in the form of concentric rings. The hollow fibers may be made up of a resorbable or non-resorbable membrane. For example, suitable components of the hollow fibers include polydioxanone, polylactide, polyglactin, polyglycolic acid, polylactic acid, polyglycolic acid/trimethylene carbonate, cellulose, methylcellulose, cellulosic polymers, cellulose ester, regenerated cellulose, pluronic, collagen, elastin, and mixtures thereof.

The bioreactor may be primed prior to seeding of the cells. The priming may comprise flushing with a buffer, such as PBS. The priming may also comprise coating the bioreactor with an extracellular matrix protein, such as fibronectin. The bioreactor may then be washed with media, such as alpha MEM.

The MSCs may be seeded in the bioreactor at a density of about 100-1,000 cells/cm², such as about 150 cells/cm², about 200 cells/cm², about 250 cells/cm², about 300 cells/cm², such as about 350 cells/cm², such as about 400 cells/cm², such as about 450 cells/cm², such as about 500 cells/cm², such as about 550 cells/cm², such as about 600 cells/cm², such as about 650 cells/cm², such as about 700 cells/cm², such as about 750 cells/cm², such as about 800 cells/cm², such as about 850 cells/cm², such as about 900 cells/cm², such as about 950 cells/cm², or about 1000 cells/cm². Particularly, the cells may be seeded at a cell density of about 400-500 cells/cm², such as about 450 cells/cm².

The total number of cells seeded in the bioreactor may be about 1.0×10⁶ to about 1.0×10⁸ cells, such as about 1.0×10⁶ to 5.0.0×10⁶, 5.0×10⁶ to 1.0×10⁷, 1.0×10⁷ to 5.0×10⁷, 5.0×10⁷ to 1.0×10⁸ cells. In particular aspects, the total number of cells seeded in the bioreactor are about 1.0×10⁷ to about 3.0×10⁷, such as about 2.0×10⁷ cells.

The cells may be seeded in any suitable cell culture media, many of which are commercially available. Exemplary media include DMEM, RPMI, MEM, Media 199, HAMS and the like. In one embodiment, the media is alpha MEM media, particularly alpha MEM supplemented with L-glutamine The media may be supplemented with one or more of the following: growth factors, cytokines, hormones, or B27, antibiotics, vitamins and/or small molecule drugs. Particularly, the media may be serum-free.

In some embodiments the cells may be incubated at room temperature. The incubator may be humidified and have an atmosphere that is about 5% CO₂ and about 1% O₂. In some embodiments, the CO₂ concentration may range from about 1-20%, 2-10%, or 3-5%. In some embodiments, the O₂ concentration may range from about 1-20%, 2-10%, or 3-5%.

### III. METHODS OF USE

The expanded MSCs of the present disclosure have broad application in treating and ameliorating disease and injury. The expanded MSCs of the present disclosure are useful in many therapeutic applications including repairing, reconstituting, and regenerating tissue as well as gene delivery. The MSCs of the present disclosure can comprise both lineage-committed and uncommitted cells; thus, both cell types can be used together to accomplish multiple therapeutic goals, even simultaneously in some embodiments. For example, in some embodiments, the expanded MSCs of the present disclosure can be used directly as stem cell transplants or be used in stem cell grafts either in suspension or on a cell culture support scaffold as noted herein above.

Certain embodiments of the present disclosure concern methods for the use of the MSCs provided herein for treating or preventing an inflammatory or immune-mediated disorder. The method includes administering to the subject a therapeutically effective amount of the MSCs, thereby treating or preventing the inflammatory or immune-mediated disorder in the subject.

The MSCs generated according to the present methods have many potential uses, including experimental and therapeutic uses. In particular, it is envisaged that such cell populations will be extremely useful in suppressing undesirable or inappropriate immune responses.

In one embodiment, a subject suffering from an autoimmune disease or an inflammatory disease is administered MSCs provided herein. In one embodiment, the subject has an autoimmune disease. Non-limiting examples of autoimmune diseases include: alopecia areata, ankylosing spondylitis, antiphospholipid syndrome, autoimmune Addison's disease, autoimmune diseases of the adrenal gland, autoimmune hemolytic anemia, autoimmune hepatitis, autoimmune oophoritis and orchitis, autoimmune thrombocytopenia, Behcet's disease, bullous pemphigoid, cardiomyopathy, celiac spate-dermatitis, chronic fatigue immune dysfunction syndrome (CFIDS), chronic inflammatory demyelinating polyneuropathy, Churg-Strauss syndrome, cicatrical pemphigoid, CREST syndrome, cold agglutinin disease, Crohn's disease, discoid lupus, essential mixed cryoglobulinemia, fibromyalgia-fibromyositis, glomerulonephritis, Graves' disease, Guillain-Barre, Hashimoto's thyroiditis, idiopathic pulmonary fibrosis, idiopathic thrombocytopenia purpura (ITP), IgA neuropathy, juvenile arthritis, lichen planus, lupus erthematosus, Meniere's disease, mixed connective tissue disease, multiple sclerosis, type 1 or immune-mediated diabetes mellitus, myasthenia gravis, nephrotic syndrome (such as minimal change disease, focal glomerulosclerosis, or mebranous nephropathy), pemphigus vulgaris, pernicious anemia, polyarteritis nodosa, polychondritis, polyglandular syndromes, polymyalgia rheumatica, polymyositis and dermatomyositis, primary agammaglobulinemia, primary biliary cirrhosis, psoriasis, psoriatic arthritis, Raynaud's phenomenon, Reiter's syndrome, Rheumatoid arthritis, sarcoidosis, scleroderma, Sjogren's syndrome, stiff-man syndrome, systemic lupus erythematosus, lupus erythematosus, ulcerative colitis, uveitis, vasculitides (such as polyarteritis nodosa, takayasu arteritis, temporal arteritis/giant cell arteritis, or dermatitis herpetiformis vasculitis), vitiligo, and Wegener's granulomatosis. Thus, some examples of an autoimmune disease that can be treated using the methods disclosed herein include, but are not limited to, multiple sclerosis, rheumatoid arthritis, systemic lupus erythematosis, type I diabetes mellitus, Crohn's disease; ulcerative colitis, myasthenia gravis, glomerulonephritis, ankylosing spondylitis, vasculitis, or psoriasis. The subject can also have an allergic disorder such as Asthma.

In yet another embodiment, the subject is the recipient of a transplanted organ or stem cells and expanded MSCs are used to prevent and/or treat rejection. In particular embodiments, the subject has or is at risk of developing graft versus host disease. GVHD is a possible complication of any transplant that uses or contains stem cells from either a related or an unrelated donor. There are two kinds of GVHD, acute and chronic. Acute GVHD appears within the first three months following transplantation. Signs of acute GVHD include a reddish skin rash on the hands and feet that may spread and become more severe, with peeling or blistering skin. Acute GVHD can also affect the stomach and intestines, in which case cramping, nausea, and diarrhea are present. Yellowing of the skin and eyes (jaundice) indicates that acute GVHD has affected the liver. Chronic GVHD is ranked based on its severity: stage/grade 1 is mild; stage/grade 4 is severe. Chronic GVHD develops three months or later following transplantation. The symptoms of chronic GVHD are similar to those of acute GVHD, but in addition, chronic GVHD may also affect the mucous glands in the eyes, salivary glands in the mouth, and glands that lubricate the stomach lining and intestines. Examples of a transplanted organ include a solid organ transplant, such as kidney, liver, skin, pancreas, lung and/or heart, or a cellular transplant such as islets, hepatocytes, myoblasts, bone marrow, or hematopoietic or other stem cells. The transplant can be a composite transplant, such as tissues of the face. MSCs can be administered prior to transplantation, concurrently with transplantation, or following transplantation. In some embodiments, the MSCs are administered prior to the transplant, such as at least 1 hour, at least 12 hours, at least 1 day, at least 2 days, at least 3 days, at least 4 days, at least 5 days, at least 6 days, at least 1 week, at least 2 weeks, at least 3 weeks, at least 4 weeks, or at least 1 month prior to the transplant. In one specific, non-limiting example, administration of the therapeutically effective amount of MSCs occurs 3-5 days prior to transplantation.

In a further embodiment, administration of a therapeutically effective amount of MSCs to a subject treats or inhibits inflammation in the subject. Thus, the method includes administering a therapeutically effective amount of MSCs to the subject to inhibit the inflammatory process. Examples of inflammatory disorders include, but are not limited to, asthma, encephalitis, inflammatory bowel disease, chronic obstructive pulmonary disease (COPD), allergic disorders, septic shock, pulmonary fibrosis, undifferentiated spondyloarthropathy, undifferentiated arthropathy, arthritis, inflammatory osteolysis, and chronic inflammation resulting from chronic viral or bacterial infections. The methods disclosed herein can also be used to treat allergic disorders.

Administration of MSCs can be utilized whenever immunosuppression or inhibition of inflammation is desired, for example, at the first sign or symptoms of a disease or inflammation. These may be general, such as pain, edema, elevated temperature, or may be specific signs or symptoms related to dysfunction of affected organ(s). For example, in renal transplant rejection there may be an elevated serum creatinine level, whereas in GVHD, there may be a rash, and in asthma, there may be shortness of breath and wheezing.

Administration of MSCs can also be utilized to prevent immune-mediated disease in a subject of interest. For example, MSCs can be administered to a subject that will be a transplant recipient prior to the transplantation. In another example, MSCs are administered to a subject receiving allogeneic bone marrow transplants without T cell depletion. In a further example, MSCs can be administered to a subject with a family history of diabetes. In other example, MSCs are administered to a subject with asthma in order to prevent an asthma attack. In some embodiments, a therapeutically effective amount of MSCs is administered to the subject in advance of a symptom. The administration of the MSCs may result in decreased incidence or severity of subsequent immunological event or symptom (such as an asthma attack), or improved patient survival, compared to patients who received other therapy not including regulatory cells.

The effectiveness of treatment can be measured by many methods known to those of skill in the art. In one embodiment, a white blood cell count (WBC) is used to determine the responsiveness of a subject's immune system. A WBC measures the number of white blood cells in a subject. Using methods well known in the art, the white blood cells in a subject's blood sample are separated from other blood cells and counted. Normal values of white blood cells are about 4,500 to about 10,000 white blood cells/μl. Lower numbers of white blood cells can be indicative of a state of immunosuppression in the subject.

In another embodiment, immunosuppression in a subject may be determined using a T-lymphocyte count. Using methods well known in the art, the white blood cells in a subject's blood sample are separated from other blood cells. T-lymphocytes are differentiated from other white blood cells using standard methods in the art, such as, for example, immunofluorescence or FACS. Reduced numbers of T-cells, or a specific population of T-cells can be used as a measurement of immunosuppression. A reduction in the number of T-cells, or in a specific population of T-cells, compared to the number of T-cells (or the number of cells in the specific population) prior to treatment can be used to indicate that immunosuppression has been induced.

In other examples, to assess inflammation, neutrophil infiltration at the site of inflammation can be measured. In order to assess neutrophil infiltration myeloperoxidase activity can be measured. Myeloperoxidase is a hemoprotein present in azurophilic granules of polymorphonuclear leukocytes and monocytes. It catalyzes the oxidation of halide ions to their respective hypohalous acids, which are used for microbial killing by phagocytic cells. Thus, a decrease in myeloperoxidase activity in a tissue reflects decreased neutrophil infiltration, and can serve as a measure of inhibition of inflammation.

In another example, effective treatment of a subject can be assayed by measuring cytokine levels in the subject. Cytokine levels in body fluids or cell samples are determined by conventional methods. For example, an immunospot assay, such as the enzyme-linked immunospot or “ELISPOT” assay, can be used. The immunospot assay is a highly sensitive and quantitative assay for detecting cytokine secretion at the single cell level. Immunospot methods and applications are well known in the art and are described, for example, in EP 957359. Variations of the standard immunospot assay are well known in the art and can be used to detect alterations in cytokine production in the methods of the disclosure (see, for example, U.S. Pat. Nos. 5,939,281 and 6,218,132).

Therapeutically effective amounts of MSCs can be administered by a number of routes, including parenteral administration, for example, intravenous, intraperitoneal, intramuscular, intrasternal, intracardiac, or intraarticular injection, or infusion.

The therapeutically effective amount of MSCs for use in inducing immunosuppression or treating or inhibiting inflammation is that amount that achieves a desired effect in a subject being treated. For instance, this can be the amount of MSCs necessary to inhibit advancement, or to cause regression of an autoimmune or alloimmune disease, or which is capable of relieving symptoms caused by an autoimmune disease, such as pain and inflammation. It can be the amount necessary to relieve symptoms associated with inflammation, such as pain, edema and elevated temperature. It can also be the amount necessary to diminish or prevent rejection of a transplanted organ.

The MSCs can be administered in treatment regimens consistent with the disease, for example a single or a few doses over one to several days to ameliorate a disease state or periodic doses over an extended time to inhibit disease progression and prevent disease recurrence. The precise dose to be employed in the formulation will also depend on the route of administration, and the seriousness of the disease or disorder, and should be decided according to the judgment of the practitioner and each patient's circumstances. The therapeutically effective amount of MSCs will be dependent on the subject being treated, the severity and type of the affliction, and the manner of administration. In some embodiments, doses that could be used in the treatment of human subjects range from at least 3.8×10⁴, at least 3.8×10⁵, at least 3.8×10⁶, at least 3.8×10⁷, at least 3.8×10⁸, at least 3.8×10⁹, or at least 3.8×10¹⁰ regulatory cells/m². In a certain embodiment, the dose used in the treatment of human subjects ranges from about 3.8×10⁹ to about 3.8×10¹⁰ regulatory cells/m². In additional embodiments, a therapeutically effective amount of MSCs can vary from about 5×10⁶ cells per kg body weight to about 7.5×10⁸ cells per kg body weight, such as about 2×10⁷ cells to about 5×10⁸ cells per kg body weight, or about 5×10⁷ cells to about 2×10⁸ cells per kg body weight. The exact amount of MSCs is readily determined by one of skill in the art based on the age, weight, sex, and physiological condition of the subject. Effective doses can be extrapolated from dose-response curves derived from in vitro or animal model test systems.

The expanded MSCs of the present disclosure can be placed in a carrier medium before administration. For infusion, expanded MSCs of the present disclosure can be administered in any physiologically acceptable medium, intravascularly, including intravenously, although they may also be introduced into other convenient sites such as into the bone marrow, where the cells may find an appropriate site for regeneration and differentiation. Usually, at least about 1×10⁵ cells/kg, at least about 5×10⁵ cells/kg, at least about 1×10⁶ cells/kg, at least about 2×10⁶ cells/kg, at least about 3×10⁶ cells/kg, at least about 4×10⁶ cells/kg, at least about 5×10⁶ cells/kg, at least about 6×10⁶ cells/kg, at least about 7×10⁶ cells/kg, at least about 8×10⁶ cells/kg, at least about 9×10⁶ cells/kg, at least about 10×10⁶ cells/kg, or more will be administered. See, for example, Ballen et al. (2001) Transplantation 7:635-645. The MSCs may be introduced by any method including injection, catheterization, or the like. If desired, additional drugs or growth factors can be co-administered. Drugs of interest include 5-fluorouracil and growth factors including cytokines such as IL-2, IL-3, G-CSF, M-CSF, GM-CSF, IFNγ, and erythropoietin. In addition, the MSCs can be injected with collagen, Matrigel, alone or with other hydrogels.

In one embodiment, the expanded MSC population of the present disclosure can be used to repair or reconstitute damaged or diseased mesenchymal tissues, such as the heart, the pancreas, the liver, fat tissue, bone, cartilage, endothelium, nerves, astrocytes, dermis, and the like. Once the expanded MSCs migrate to or are placed at the site of injury, they can differentiate to form new tissues and supplement organ function. In some embodiments, the cells are used to promote vascularization and, therefore, improve oxygenation and waste removal from tissues. In these embodiments, the expanded MSCs of the present disclosure can be used to increase function of differentiated tissues and organs such as the ischemic heart as in cardiac failure or ischemic nerves as in stroke.

The expanded MSCs of the present disclosure can also be used for gene therapy in patients in need thereof. In some embodiments, more mature lineage-committed cells will be useful, especially where transient gene expression is needed or where gene transduction is facilitated by the maturation and division of the cells. For example, some retroviral vectors require that the cell be cycling for the gene to be integrated. Methods for transducing stem and progenitor cells to deliver new and therapeutic genes are known in the art.

Administered MSCs may also comprise a mixture of cells herein described and additional cells of interest. Additional cells of interest include, without limitation, differentiated liver cells, differentiated cardiac muscle, differentiated pancreatic cells, and the like.

The expanded MSCs may be administered in combination with one or more other therapeutic agents for the treatment of the immune-mediated disorder. Combination therapies can include, but are not limited to, one or more anti-microbial agents (for example, antibiotics, anti-viral agents and anti-fungal agents), anti-tumor agents (for example, fluorouracil, methotrexate, paclitaxel, fludarabine, etoposide, doxorubicin, or vincristine), immune-depleting agents (for example, fludarabine, etoposide, doxorubicin, or vincristine), immunosuppressive agents (for example, azathioprine, or glucocorticoids, such as dexamethasone or prednisone), anti-inflammatory agents (for example, glucocorticoids such as hydrocortisone, dexamethasone or prednisone, or non-steroidal anti-inflammatory agents such as acetylsalicylic acid, ibuprofen or naproxen sodium), cytokines (for example, interleukin-10 or transforming growth factor-beta), hormones (for example, estrogen), or a vaccine. In addition, immunosuppressive or tolerogenic agents including but not limited to calcineurin inhibitors (e.g. cyclosporin and tacrolimus); mTOR inhibitors (e.g. Rapamycin); mycophenolate mofetil, antibodies (e.g. recognizing CD3, CD4, CD40, CD154, CD45, IVIG, or B cells); chemotherapeutic agents (e.g. Methotrexate, Treosulfan, Busulfan); irradiation; or chemokines, interleukins or their inhibitors (e.g. BAFF, IL-2, anti-IL-2R, IL-4, JAK kinase inhibitors) can be administered. Such additional pharmaceutical agents can be administered before, during, or after administration of the regulatory B cells, depending on the desired effect. This administration of the cells and the agent can be by the same route or by different routes, and either at the same site or at a different site.

### IV. KITS

In some embodiments, a kit that can include, for example, one or more media and components for the production of MSCs is provided. Such formulations may comprise a cocktail of factors, in a form suitable for combining with MSCs. The reagent system may be packaged either in aqueous media or in lyophilized form, where appropriate. The container means of the kits will generally include at least one vial, test tube, flask, bottle, syringe or other container means, into which a component may be placed, and preferably, suitably aliquoted. Where there is more than one component in the kit, the kit also will generally contain a second, third or other additional container into which the additional components may be separately placed. However, various combinations of components may be comprised in a vial. The components of the kit may be provided as dried powder(s). When reagents and/or components are provided as a dry powder, the powder can be reconstituted by the addition of a suitable solvent. It is envisioned that the solvent may also be provided in another container means. The kits also will typically include a means for containing the kit component(s) in close confinement for commercial sale. Such containers may include injection or blow molded plastic containers into which the desired vials are retained. The kit can also include instructions for use, such as in printed or electronic format, such as digital format.

### V. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1—Expansion of Mesenchymal Stromal Cells

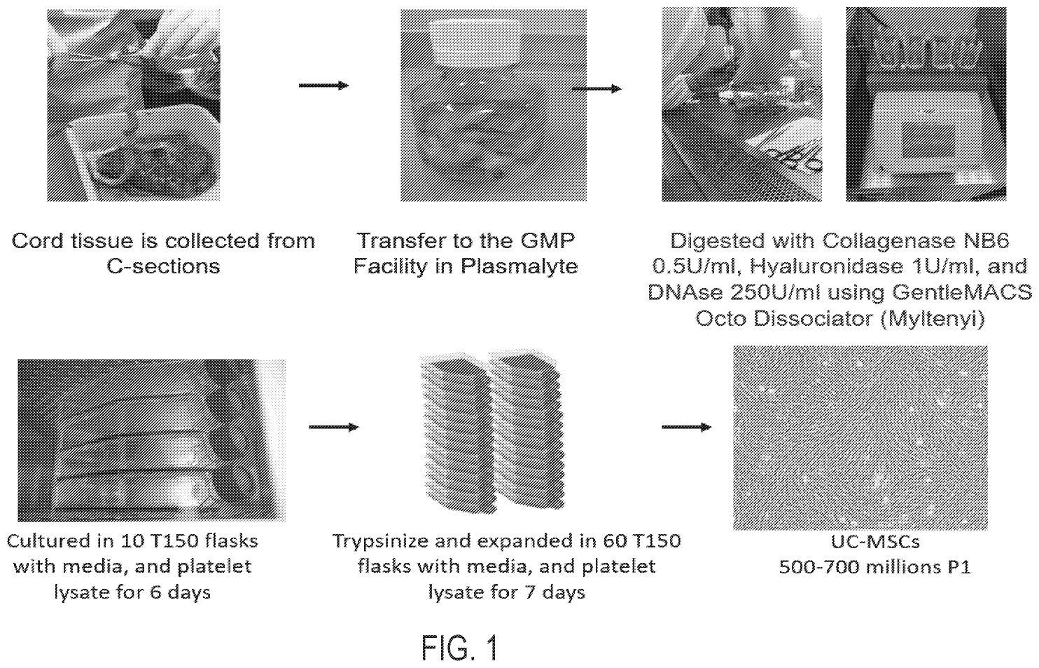

The Terumo Quantum Cell Expansion system (bioreactor used) is an automated hollow fiber cell culture platform designed for GMP compatible production of cells. Briefly, cord tissue was obtained from normal infant deliveries and a hyaluronidase containing enzyme cocktail (Collagenase NB6 0.5 U/ml, Hyaluronidase 1 U/ml, and DNAse 250 U/ml) was used to digest the tissue. Following digestion the cells were plated in flasks and cultured for several days. When the cells were ˜85% confluent they were trypsinized, replated and cultured again and when confluent removed and frozen as passage 1 (P1). (FIG. 1).

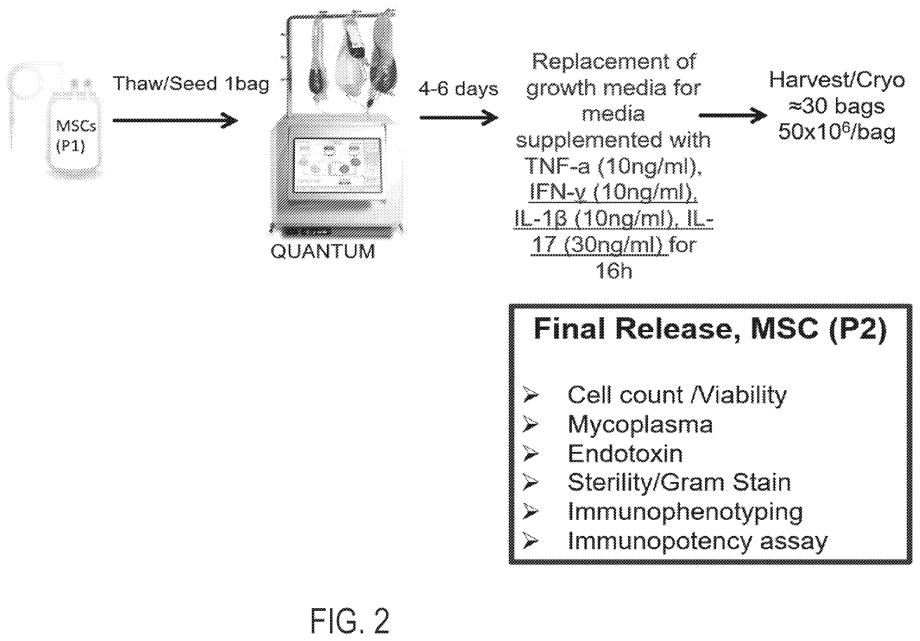

The P1 cells were then thawed and expanded in the Quantum Bioreactor for 4-6 days (until reaching confluency). Once the ideal confluency was identified based on the glucose and lactate levels in the bioreactor, the cells were pre-activated with the present combination of cytokines including TNF, IFN-γ, IL-1β, and IL-17 for 16 hours, washed, harvested and evaluated in various assays or frozen for clinical use (FIG. 2).

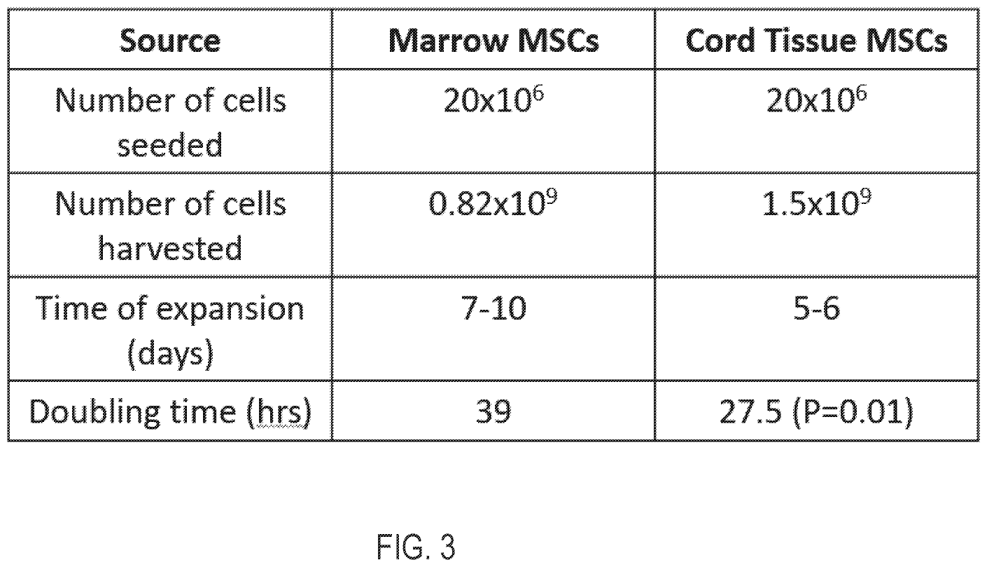

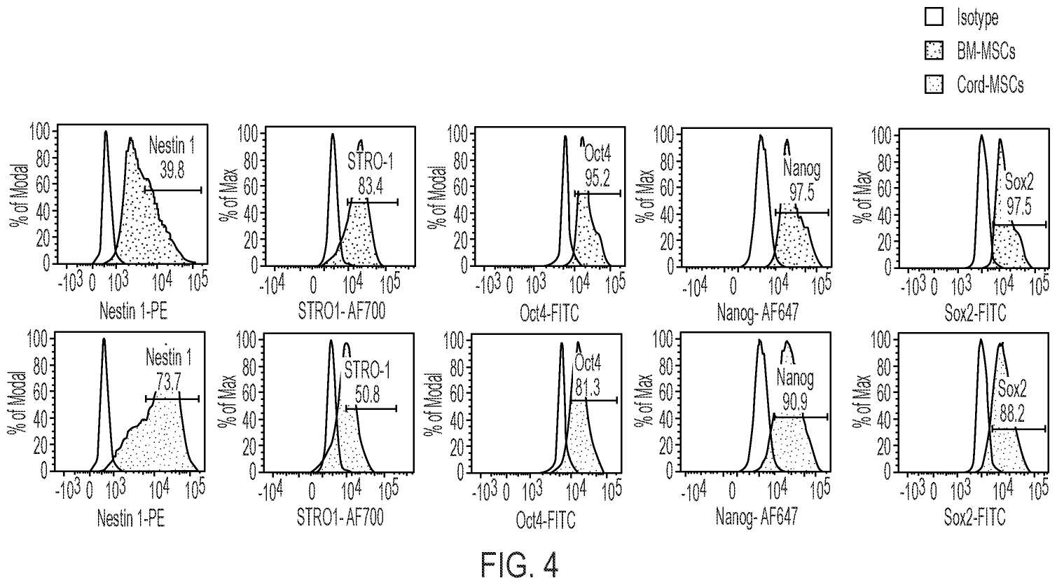

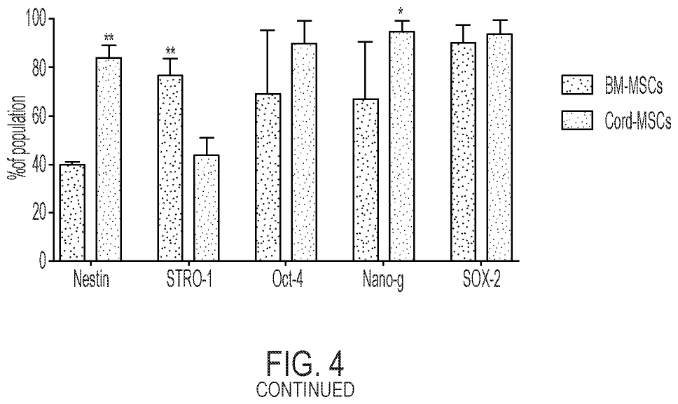

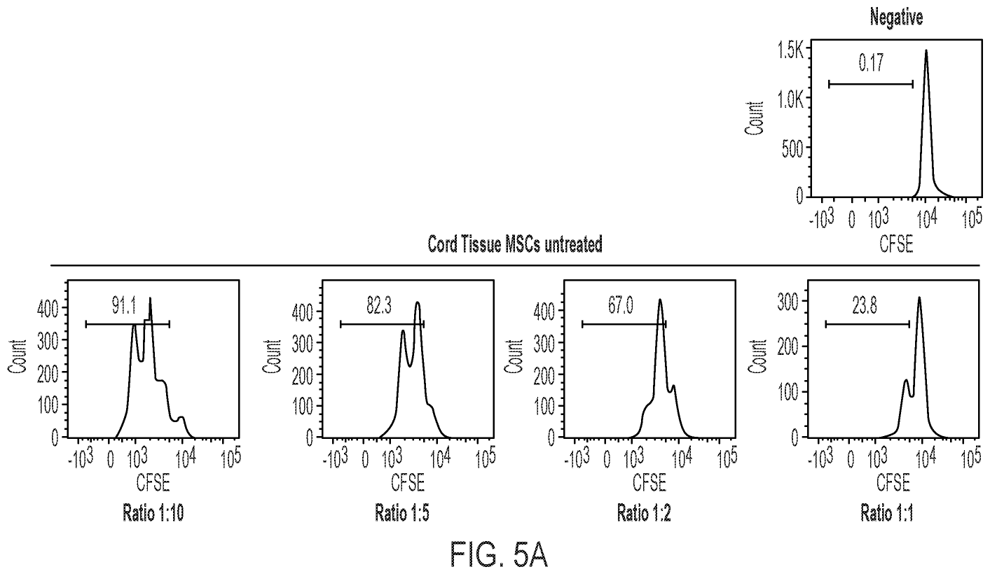

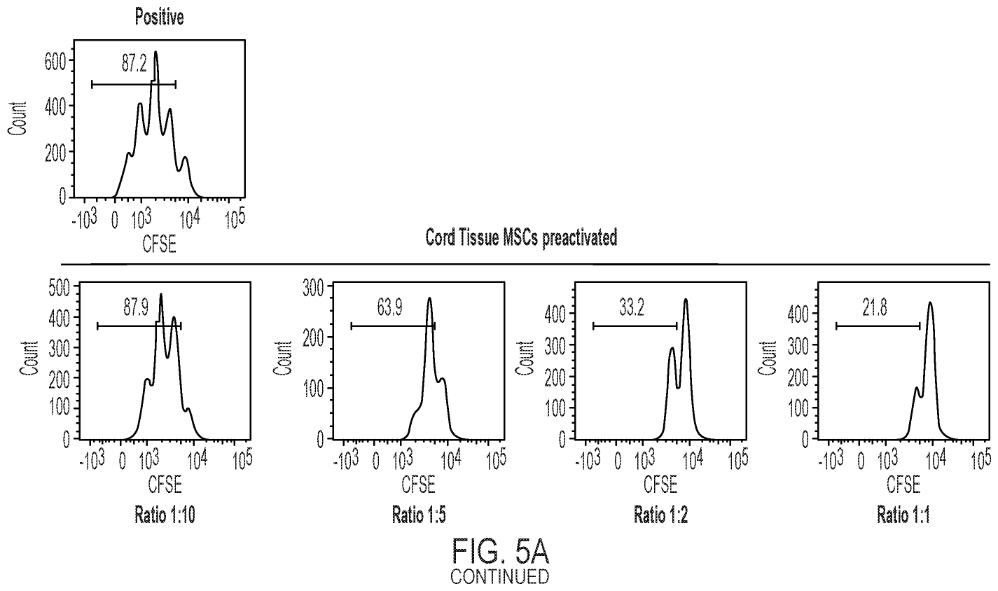

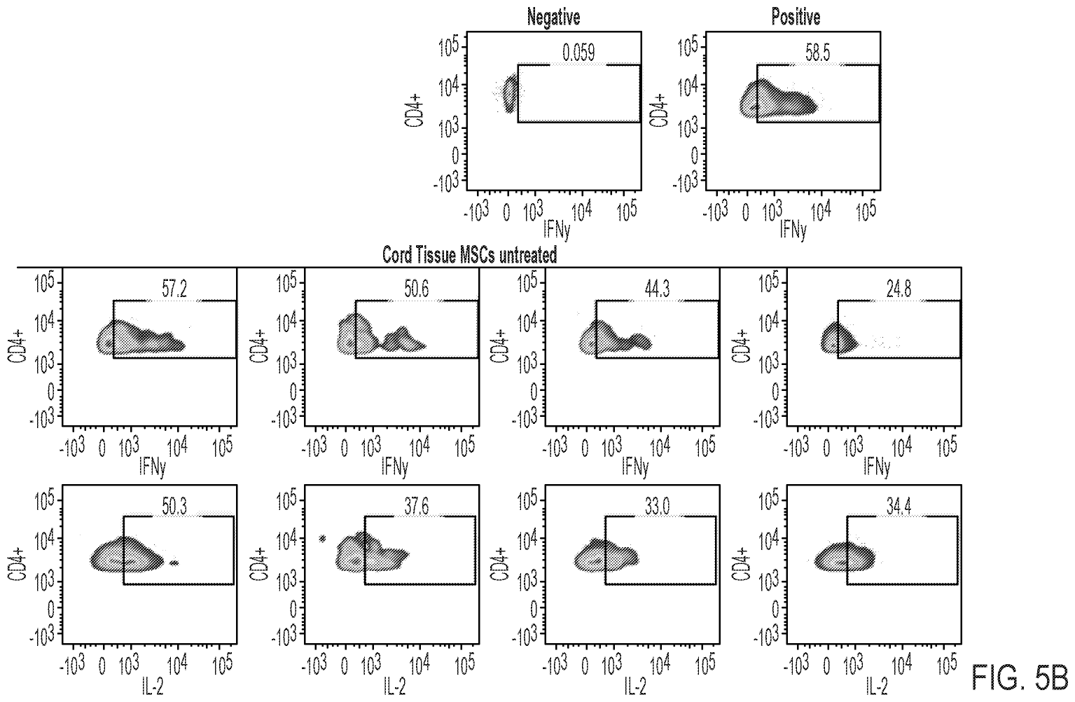

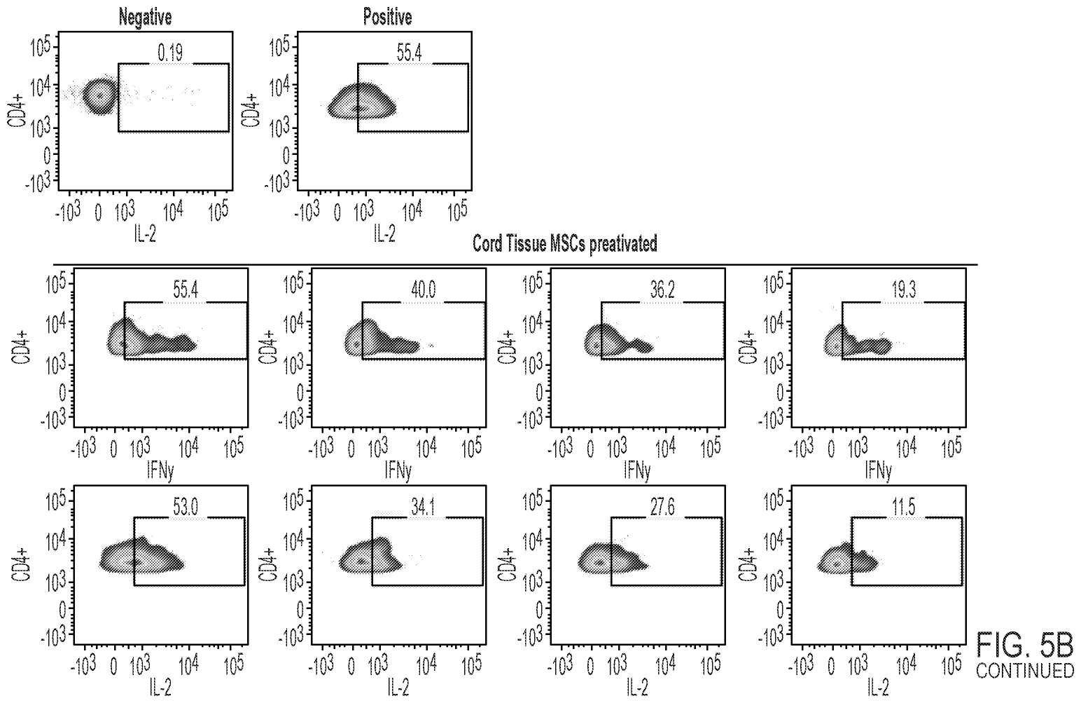

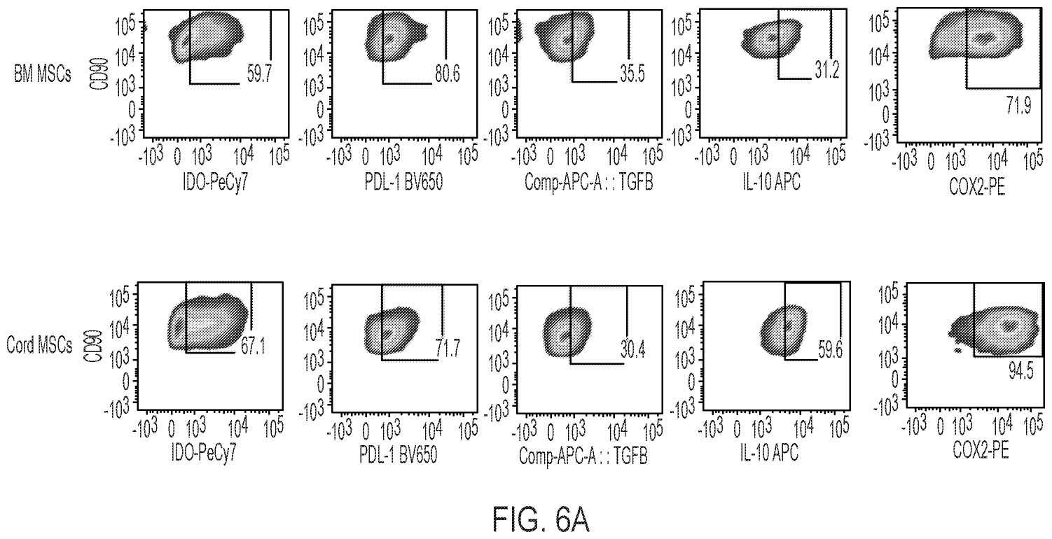

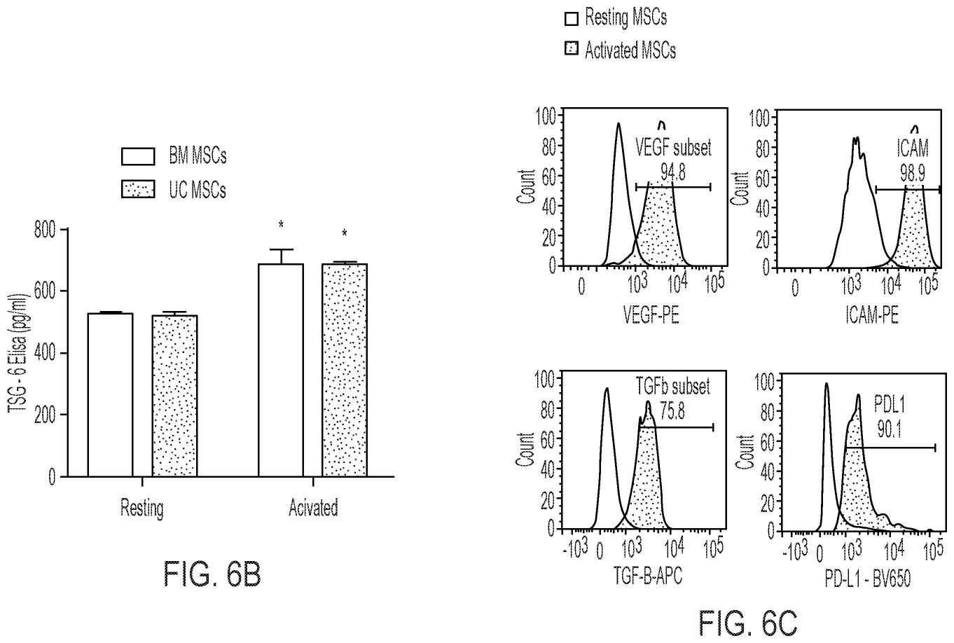

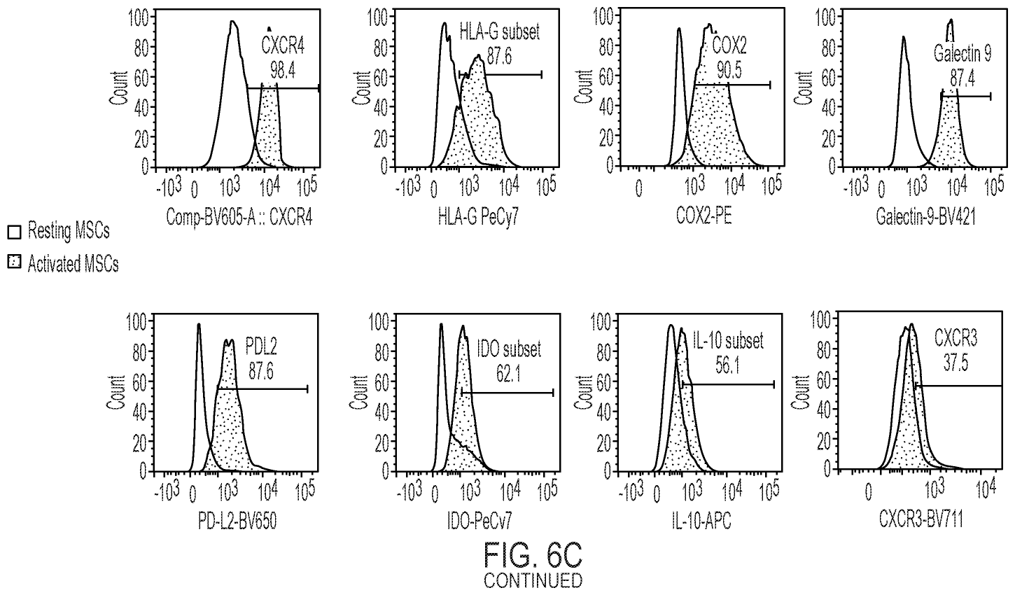

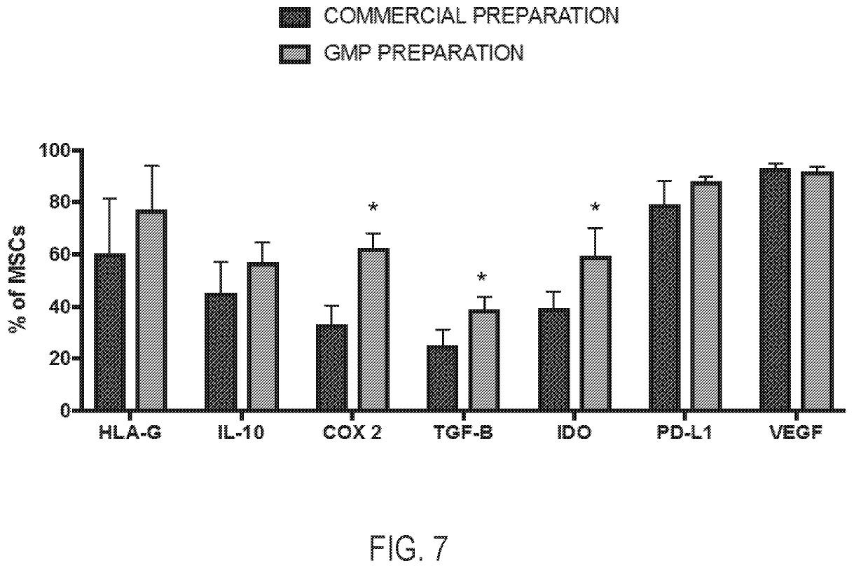

When 20 million cord tissue versus bone marrow derived MSCs were added to the bioreactor, the number of MSCs generated with cord tissue was almost twice that of bone marrow-derived MSCS in a shorter period of time (FIG. 3). The CBt-MSCs expressed significantly higher numbers of the “stemness” markers Nestin, Stro-1, Oct-4, Nanog and Cox-2 (FIG. 5). Importantly, the pre-activated CBt-MSCs were more suppressive than baseline (not activated) CBt-MSCs or BM-MSCs (FIG. 5). They expressed higher levels of the anti-apoptosis factors (VGEF, TGFβ)), anti-inflammatory/antiproliferative factors (TSG-6), immunomodulatory factors (PD-L1, IDO, PGE2, IL-10, TGFβ), and chemoattraction-homing factors (CXCR4, CXCR3) (FIG. 6). Thus, the pre-activated, expanded CBt-MSCs were generated efficiently in large clinically relevant doses and had a greater therapeutic effect than other MSCs preparations (FIG. 7).

### Example 2—Materials and Methods

CBt was obtained from consented healthy mothers of full-term neonates following elective cesarean section. The CBt was transported in plasmalyte with penicillin/streptomycin. The CBt was cut into 7 equal fractions and incubated for 76 minutes in 37° C. in C-Tubes (Miltenyi) with various enzyme combinations including collagenase-NB4/6 (Serva) and hyaluronidase (Sigma Aldrich) with or without DNase (Genentech) in the GentleMACS Octo Dissociator (Miltenyi). The cell suspension was filtered, washed and resuspended in alpha-MEM media containing 10% Platelet lysate, L-glutamine, heparin (complete media) with pen-strep and seeded into T175 flasks, then cultured until the MSCs were 80% confluent. The cells were harvested and expanded to P1 in T175 flasks to 80% confluence using complete media without antibiotics.

After harvest of P1, the MSCs were analyzed for the expression of the typical MSC surface markers by flow cytometry and cryopreserved. Expansion was subsequently performed in a Quantum Bioreactor (Terumo) for 5-6 days Immunosuppressive potential of CBt-MSCs was tested in vitro by CD4⁺ T cell proliferation assay (CFSE) and CD4⁺ T cell cytokine secretion assay. Half of the CBt-MSCs were pre-activated with Interferon Gamma, then seeded into a 96 well plate and the others were seeded untreated. The following day, the MSCs were incubated with 10⁵ isolated CD4⁺ T cells at ratios of 1:1, 1:2, 1:10 and 1:20. CD3/28 beads (ThermoFisher Scientific) were added to all wells except the negative control. Isolated T cells were stained with CFSE for 10 minutes then incubated with 10% Fetal Bovine Serum (FBS) prior to co-culture with MSCs.

After 72 hours, the wells were treated with BFA (10×), PMA (100×) and ionomycin (10×). Half of the wells were harvested, washed and stained with anti-CD4 (Biolegend) and Live-Dead dye (ThermoFisher Scientific). After Cytofix/Cytoperm Fixation and Permeabilization Solution (BD Biosciences), then 1× buffer were added, cells were stained for IL-2 (BD), TNF-alpha (BD) and Interferon gamma (BD Biosciences). On day 5, the remaining wells were harvested and stained with anti-CD4-APC (Biolegend) and Live-Dead (ThermoFisher Scientific). Flow cytometry was performed on all samples using the Fortessa X20 (BD Biosciences), then analyzed with FlowJo software.

Following enzymatic digestion, the samples without DNase had poor P0 to P1 growth (less than 80% confluent by day 10) and thus were eliminated. NB4, hyaluronidase and DNase became the standard enzyme combination. After seeding the

Bioreactor with a median of 51×10E6 CBt-MSCs (range 45 to 62×10E6 cells), expansion in the Bioreactor for 5-6 days yielded a median of 1495×10E6 CBt-MSCs (range 1245 to 1935×10E6). The median doubling time (the time required for MSCS to proliferate and double in number) for CBt-MSCs was 28.2 hours (range 24.5 to 29.7) (n=3). The immunosuppression assay demonstrated that CBt MSCs inhibit the proliferation of CD4⁺ T cells in a dose-dependent manner Moreover, CBt-MSCs reduced the cytokine expression on stimulated CD4⁺ T cells (IFNγ, TNFα, IL-2) with each successive generation of CD4⁺ T-cells. Thus, the present methods provide a novel, standardized GMP-compliant protocol for the isolation of MSCs from whole CBt. Large-scale expansion of CBt-MSCs with immunosuppressive properties can be generated quickly and efficiently in the Terumo bioreactor.

### Example 3—Characterization of Mesenchymal Stem Cells

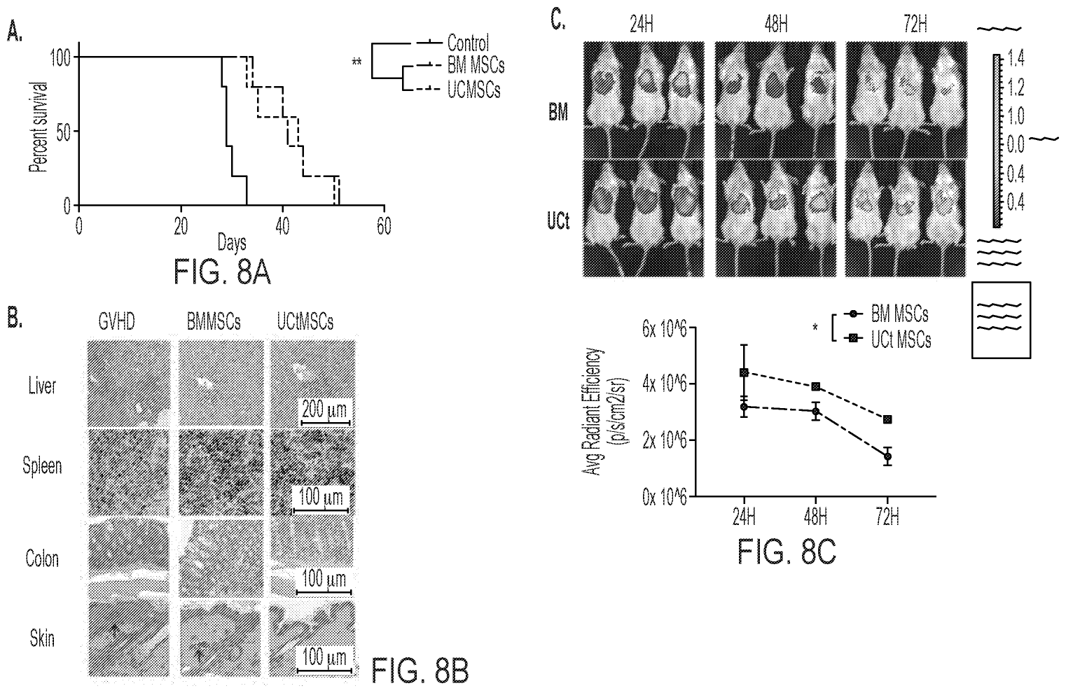

The MSC derived in Example 1 were characterized in vivo to determine their functionality. It was found that fresh CBt-derived MSCs increased the survival in a xenogeneic graft versus host disease (GVHD) mouse model (FIG. 8).

NSG (NOD.Cg-Prkdcscid IL2rgtm1Wjl/SzJ) 7 week old male mice were purchased from the Jackson Laboratory (Bar Harbor, Me., USA) and allowed to acclimate for one week before the experiments. Mice (11 week old) were sub-lethally irradiated (300 cGy) 24 hours before the transplantation of 2×10⁶ G-mobilized peripheral blood progenitor cells (PBPCs) on day 0 of the experiment. The mice then received five doses of either BM or CBt-derived MSCs in a dose of 2×10⁶ per infusion via tail vein injection on days +8, +11, +14, +18, and +21. Survival, weight loss, fur texture, physical activity, skin integrity and hunched back were recorded daily. The severity of GVHD was assessed by means of a clinical scoring system described by Cooke et al. Five mice per group were used, and experiments were performed three times. Results showed a significant increase in the overall survival of the mice who received either fresh BM or CBT-derived MSCs compared with the GVHD controls (FIG. 8A).

In some experiments either BM or CBt-MSCs were labeled using Xenolight DiR (Perkin Elmer, Rodgau, Germany), a NIR lipophilic carbocyanine dye excited at 750 nm, with an emission peak at 782 nm. Cells were resuspended in PBS (1×10⁶ cells/ml) and incubated with DiR (10 μg DiR/ml) for 30 min at 37° C. The cells were then washed 2 times with culture medium to remove non-incorporated dye. FIG. 8B shows the histopathological samples which demonstrate a slight reduction in GVHD signs of liver, spleen and colon of the mice treated (with either BM- or CBT-MSCs) compared with the GVHD control. FIGS. 8C and 8D show the short-term biodistribution experiment using DiR-immunofluorescence labeled MSCs infused via tail vein into the mice. CBt-MSCs showed a significant improvement in persistence compared to BM-MSCs over the course of 72 hours.

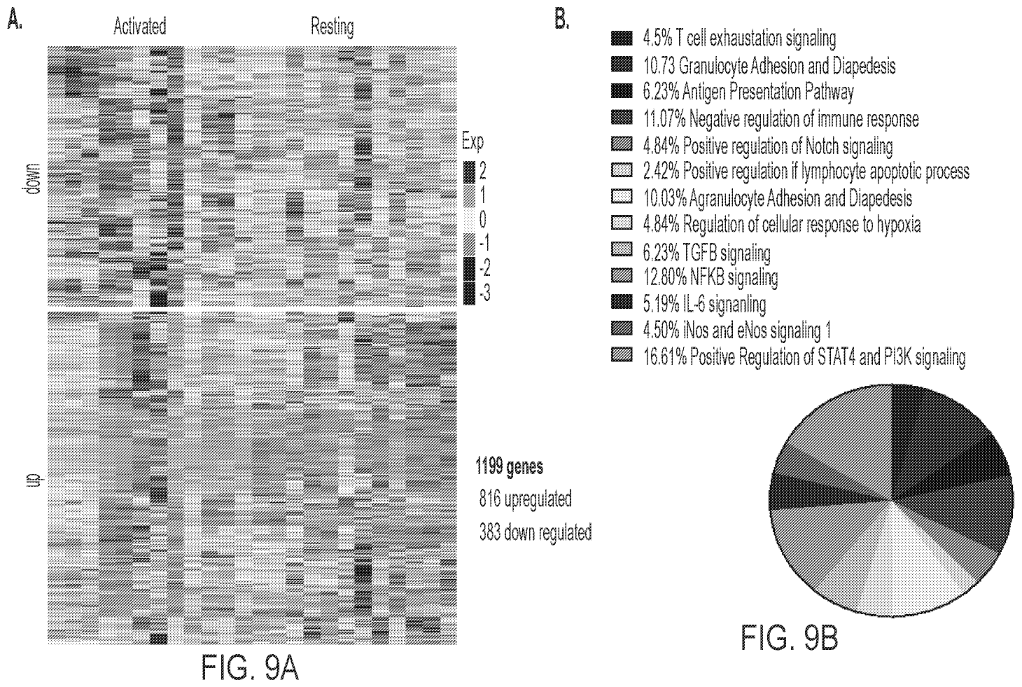

Next, it was found that the activation of CBt-MSCs revealed a unique profile with higher immunosuppressive properties (FIG. 9). CBt-MSCs were cultured using alpha MEM media supplemented with 1% L-glutamine and 5% human platelet lysate until 85% of confluence. Then, the media was replaced by activation media (alpha MEM media, supplemented with 1% L-glutamine, IFN gamma (10 ng/ml), TNF alpha (10 ng/ml), IL-1B (10 ng/ml) and IL-17 (10 ng/ml)) for 24-36 hours. After that time cells were harvested and RNA was extracted and purified (RNeasy Plus Mini Kit, Qiagen) following manufacture instructions. Twelve samples were analyzed per culture condition. After RNA extraction, cDNA preamplification and sequencing quality control, a cDNA library was prepared, and the transcriptome of these cells was sequenced on an Illumina HiSeq 2500 system. Analysis of RNAseq data was performed by MD Anderson Bioinformatics department. Sequencing reads were aligned to human reference genome (hg38) using TOPHAT2 v2.0.1346. The gene expression levels were measured by counting the mapped reads using HTSEQ47,48 based on hg38 GENCODE v25 gene model. The differentially expressed genes were identified using EdgeR package48, with FDR (false discovery rate) cutoff <0.01 and fold change >2. The network analyses were generated through the use of Ingenuity Pathway Analysis® (IPA®, Qiagen).

As summarized in FIG. 9A, a heat map of genes differentially expressed between resting and activated MSCs, showed the upregulation of 816 genes and down regulation of 383 genes in activated CBt-MSCs compared with the resting CBt-MSCS. The Ingenuity Pathway Analysis (IPA) of the genes evaluated on resting and activated UC-MSCs revealed that activation of cells induced the expression of genes associated to several immune regulatory pathways such as T cell exhaustion, negative regulation of immune response, IL-6 signaling, and induction of T cell apoptosis (FIG. 9B).

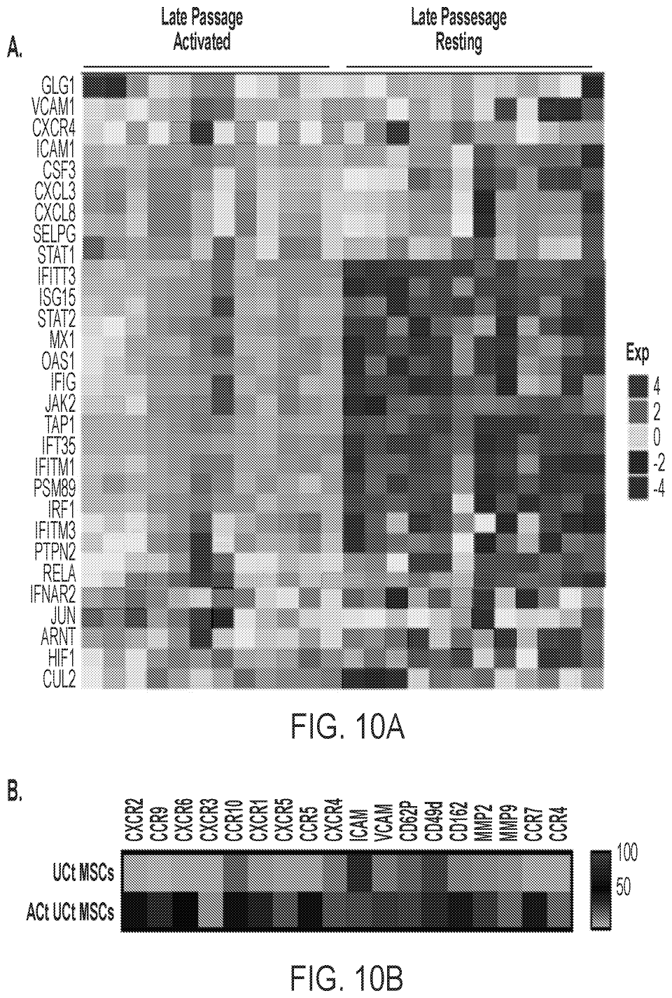

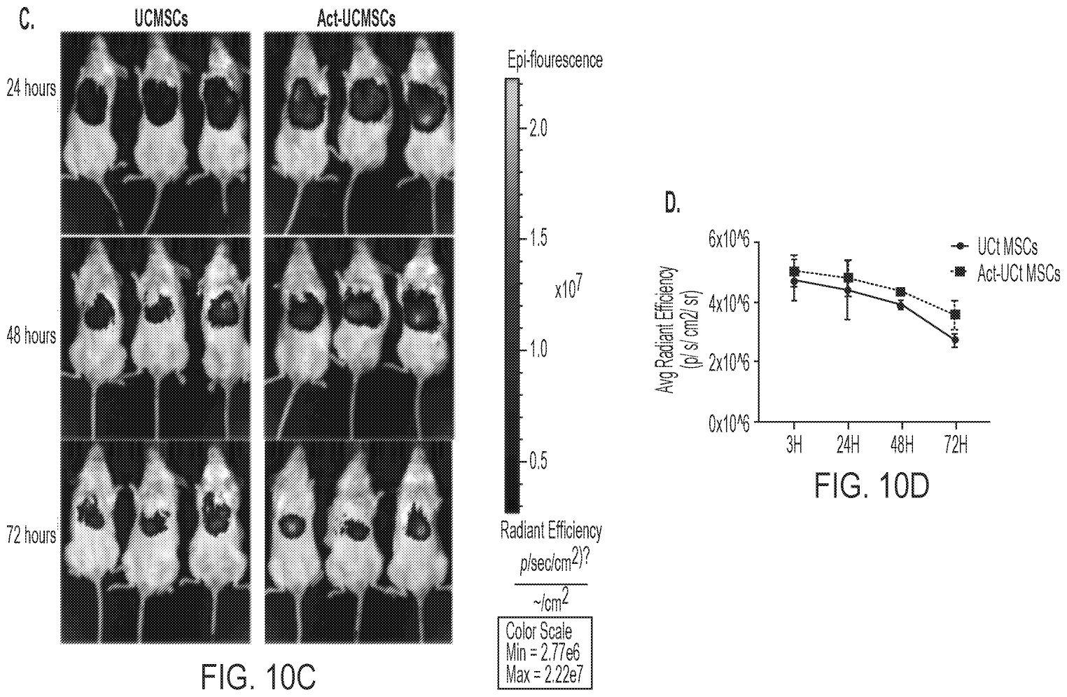

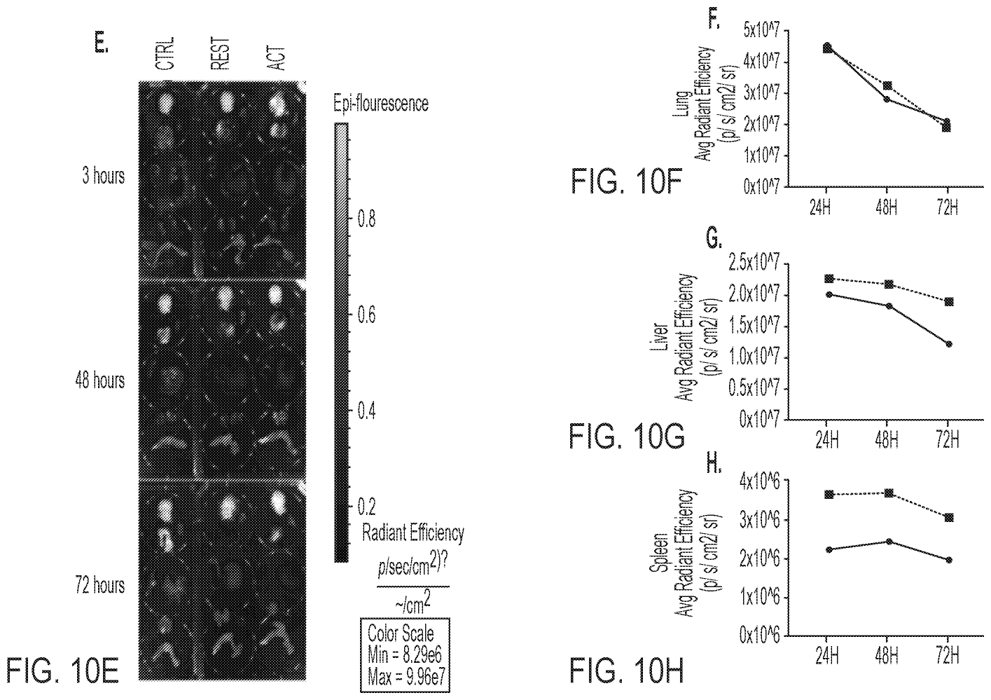

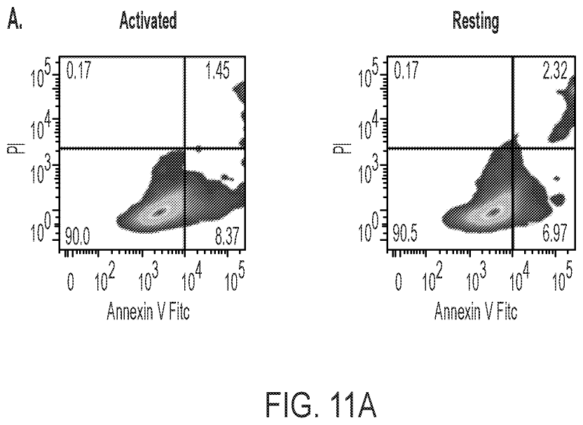

It was also observed that activation enhanced CBt-MSC homing and biodistribution in a GVHD xenograft mice model (FIG. 10). The IPA analysis performed on the RNA extracted from activated vs resting CBt-MSCs revealed the activation of several genes related with diapedesis and homing including homing receptors and key adhesion molecules related to adhesion and invasion on the activated CBt MSCs, presented in heat map (FIG. 10A). Heat map of homing receptors, adhesion molecules, and invasion proteins (metalloproteinases) on activated CBT MSCs vs. resting MSCs, which were evaluated by flow cytometry (FIG. 10B). For biodistribution experiments either resting or activated CBt-MSCs prelabeled with DiR were administered to NSG mice (2×10⁶ MSCs per mouse) via tail vein injection on day +8, post GvHD induction (PBPC infusion on Day 0).

As shown in FIGS. 10C and 10D, after 72 hours fluorescence analysis it was observed that activated CBT-MSCs persisted in the mice longer than control CBt-MSC for up to three days. As shown in FIG. 10E, the mouse tissues were then harvested at 3 h, 48 h, and 72 h post-injection and the average radiant efficiency was calculated by tissue. A trend toward higher fluorescence level was shown for the activated CBt-MSCs group compared to control MSC group, as shown in FIG. 10F for the lung, FIG. 10G for the liver, and FIG. 10H for the spleen.

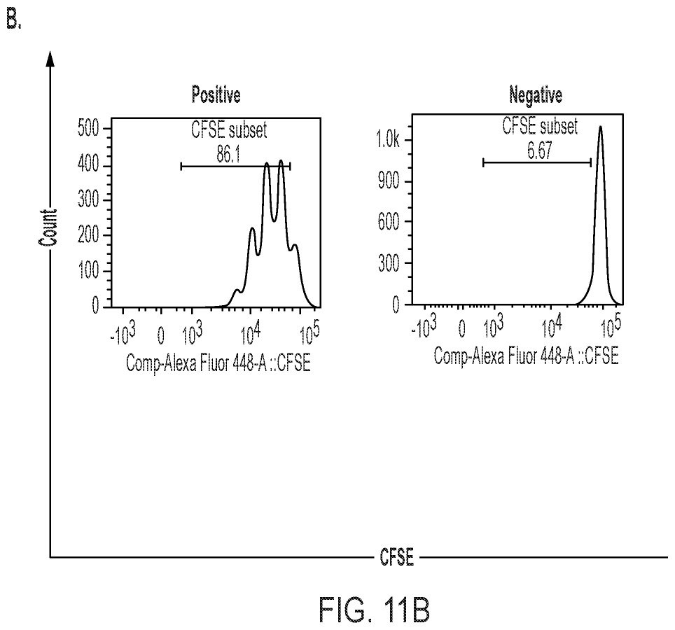

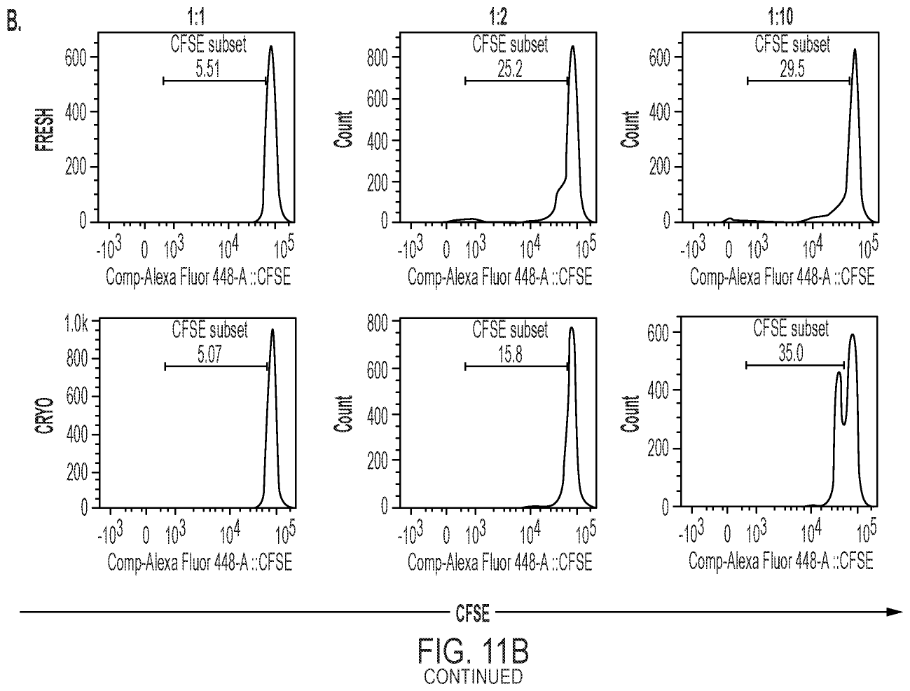

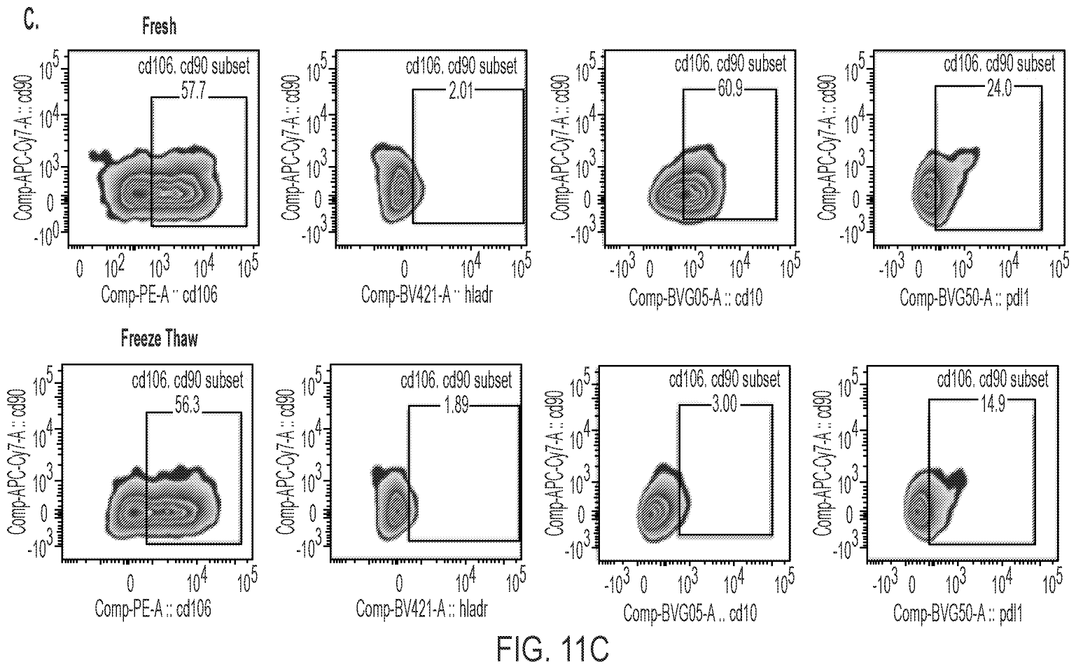

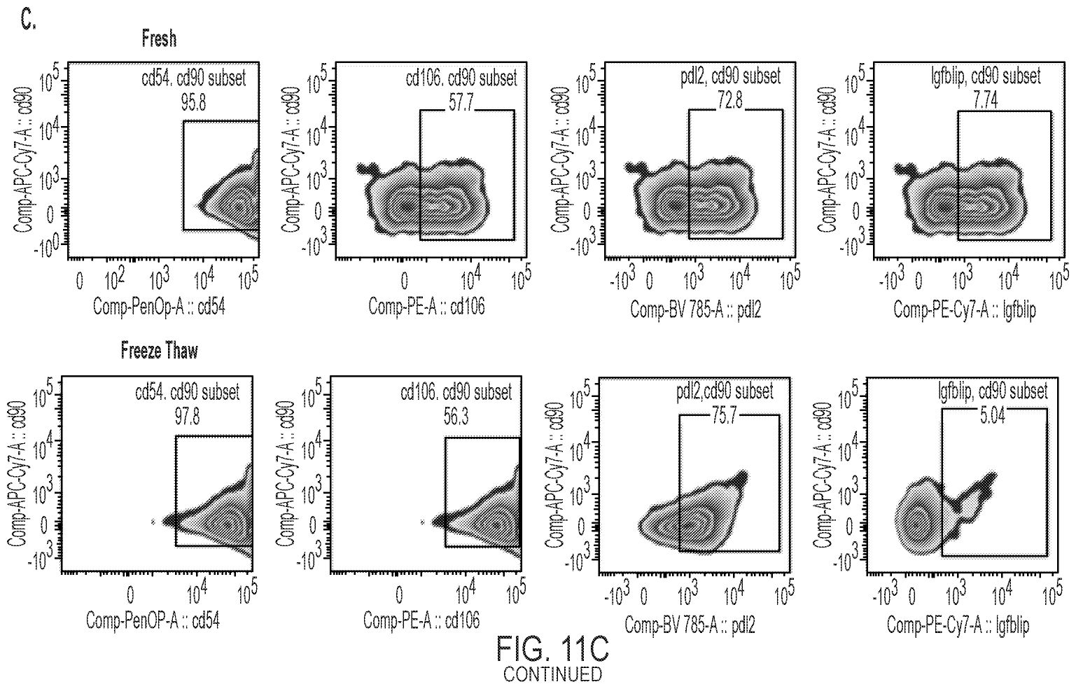

Next, it was observed that cryopreserved activated UCMSCs demonstrated a similar viability, phenotype, and efficacy controlling T cell activation compared to fresh activated UCMSCs (FIG. 11). Activated cells were harvested and frozen for 2 weeks. After that time, cells were thawed and their phenotype was analyzed using flow cytometry. FIG. 11A shows a representative FACS plot of the viability of CBt-MSCs determined by flow cytometry using annexin V and propidium iodide assay. T cell immunosuppression mediated by CBt MSCS (resting and activated) were evaluated by CFSE assay. Briefly, lymphocytes were obtained from healthy volunteers PBMNCs and isolated by ficoll. T cells were isolated using Pant T cells microbeads (Miltenic) and stained with 5(6)-carboxyfluorescein diacetate N-succinimidyl ester (CFSE; Sigma-Aldrich). They were then suspended in lymphocyte medium: RPMI 1640 medium (Gibco, Grand Island, N.Y., USA) containing 10% FBS, 1% L-glutamine, penicillin (100 units/ml), and streptomycin (100 μg/ml). For the coculture assay, 100 ul of MSCs were seeded in a 96 well plate at different concentration (1×10⁶/ml, 0.5×10⁶/ml, 1×10⁵/ml) and incubated for 1 hour. For the assay, 10⁵ lymphocytes stimulated with CD3/CD28 beads (Invitrogen) were seeded on the MSCs monolayer during 4 days. After that time, cells were harvested, washed and stained for viability (Live/Dead Aquia Fluorescence), CD3, CD8, CD4. Proliferation of T cells was evaluated by flow cytometry. Naive (unstimulated) lymphocytes (negative control) and stimulated T cells without MSCs (positive control) were used as a control. FIG. 11B shows a representative histogram of a T cell proliferation CFSE assay, demonstrating the total suppression of T cell activated with CD3/CD28 beads in all the different ratios. FIG. 11C shows a representative FACS plot of activated MSCs phenotype with either fresh or frozen/thawed cells showing the persistence of the expression of immunosuppressive factors.

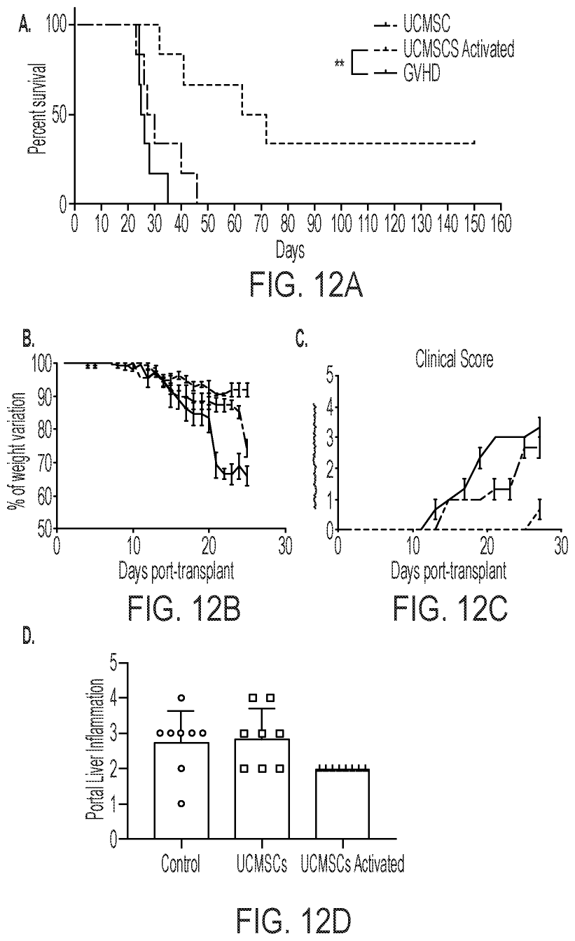

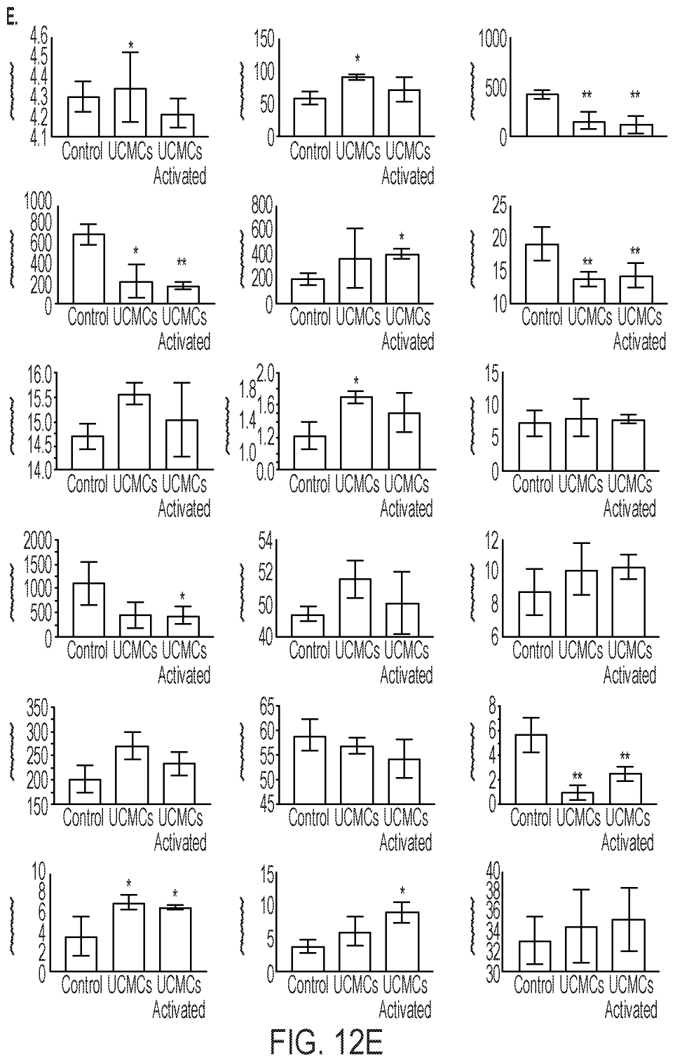

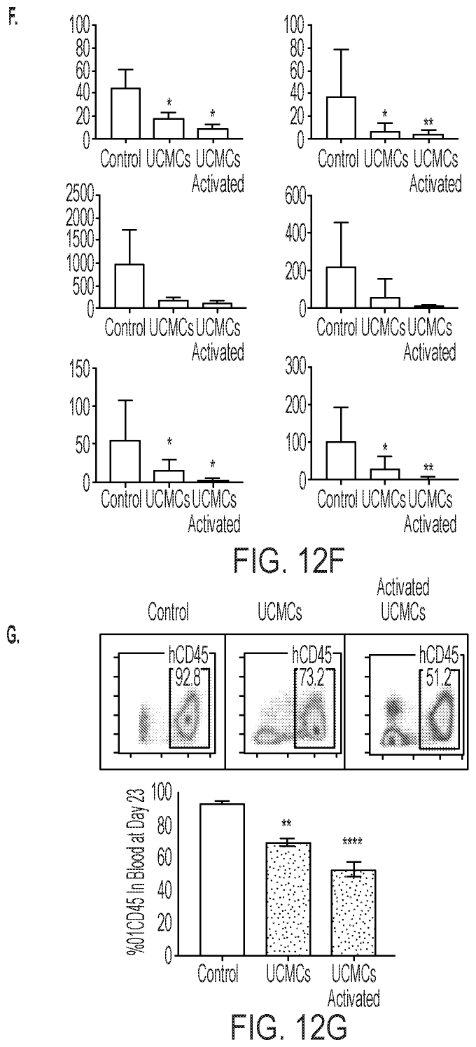

It was also observed that cryopreserved activated CBt-MSCs increased the overall survival and reduced GVHD toxicities (FIG. 12). Mice were irradiated 300 cGy, followed by infusion of 2×10⁶ G-mobilized PBPCs within 24 hours as described above. CBt-MSCs were thawed and washed twice in DPBS and resuspended in a concentration of 2×10⁶ cells/0.1 mL in saline. The CBt-MSCs were infused within 3 h of thawing for all experiments. The GVHD control mice were injected with a 0.1 mL saline solution. For this experiment 11 mice were used from each group. Three mice in each group were euthanized for histological analysis on day 24. Mice were anesthetized and blood was collected and processed. The human lymphocyte population was determined by flow cytometry. Plasma samples were analyzed to determine cytokines using the microarray assay. Assays were performed in triplicate.

FIGS. 12A-C summarize the in vivo experiment with groups of mice (n=8 mice per group). FIG. 12A shows the survival curves for the untreated (GVHD controls), recipients of unactivated CBt-MSCs, and recipients activated CBt-MSCs. The data demonstrated a survival benefit for the recipients of the activated CBT-MScs compared to unactivated MSCs or controls. FIG. 12B shows the percent weight variations demonstrating less weight loss for the recipients of activated CBt-MSCs versus the other two groups. FIG. 12C shows the average GVHD scores, again showing less GVHD for the activated CBT-MSC group. FIG. 12D shows the portal liver inflammation compared among the three groups of mice at the end time point. FIG. 12E shows the comparison of hematological tests from mice that were untreated (control), treated with resting CBt-MSCs versus the activated (activated) MSCs. The blood tests include WBC count, MCV, MCHC, Hematocrit, Hemoglobin, Platelet count, RBC count, MCH, RDW, Albumin, Alkaline phosphatase, potassium, LDH, AST, Glucose, ALT, Phosphorus, total protein. Results show an improvement in the platelet count, glucose, WBC count, and liver function (ALT, AST) in the group treated with activated MSCs compared with the control or unactivated MSC groups.

FIG. 12F shows the results of the cytokine levels in the blood of the mice, revealing that both the activated and unactivated CBt-MSCs reduced the presence of inflammatory cytokines compared to the control mice. FIG. 12G shows the percentage of human CD45 in the blood of the mice on Day 24. The left panel shows a representative FACS plot from each group, while the right panel shows a bar plot with statistical comparison compared to control group (untreated), with ** p-value<0.01, **** p-value<0.0001 demonstrating fewer human CD45⁺ cells in the blood of both MSC recipient groups with the activated MSCs showing fewer than unactivated MSC recipients.

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
Cohen et al., J Immunol. 175:5799-5808, 2005.
Current Protocols in Immunology, Ed Coligan et al, Wiley, 1994.
Czerkinsky et al., J. Immunol. Methods, 110:29-36, 1988.
EP 957359
Fast et al., Transfusion 44:282-5, 2004.
Fedorov et al., Sci. Transl. Medicine, 5(215), 2013.
He Y, et al. Journal of immunology research, 7, 2014.
Heemskerk et al. Hum Gene Ther. 19:496-510, 2008.
International Patent Publication No. WO2000/06588
International Patent Publication No. WO2000/06588
International Publication No. PCT/US95/01570
International Publication No. WO2000/06588
International Publication No. WO2005/035570
Janeway et al, Immunobiology: The Immune System in Health and Disease, 3^{rd} Ed., Current Biology Publications, p. 433, 1997.
Johnson et al. Blood 114:535-46, 2009.
Lefranc et al., Dev. Comp. Immunol. 27:55, 2003.
Li, Nat Biotechnol. 23:349-354, 2005.
Olsson et al. J. Clin. Invest. 86:981-985, 1990.
Parkhurst et al., Clin Cancer Res. 15: 169-180, 2009.
PCT Patent Publication No. WO2001/083517
Taitano et al., The Journal of Immunology, 196, 2016.
U.S. Pat. No. 7,109,304
U.S. Pat. No. 5,939,281
U.S. Pat. No. 5,939,281
U.S. Pat. No. 6,218,132
U.S. Pat. No. 6,218,132
U.S. Pat. No. 6,264,951
U.S. Pat. No. 6,426,331
U.S. Pat. No. 7,488,490
U.S. Pat. No. 7,488,490
U.S. Patent Publication No. 2007/0078113
Varela-Rohena et al., Nat Med. 14: 1390-1395, 2008.
WO2014/055668
Wong et al., Cytotherapy, 4: 65-76, 2002.

1. A method for the expansion of cord tissue-derived mesenchymal stromal cells (MSCs) comprising:
(a) obtaining a population of MSCs from cord tissue;
(b) pre-activating the MSCs in the presence of at least three cytokines selected from the group consisting of TNFα, IFNγ, IL-1β, and IL-17; and
(c) expanding the pre-activated MSCs to obtain a population of expanded MSCs.

2. The method of claim 1, wherein the population of MSCs from cord tissue were previously cryopreserved.

3. The method of claim 1, wherein the obtaining comprises treating the cord tissue with an enzyme cocktail.

4. The method of claim 3, wherein the enzyme cocktail comprises hyaluronidase and collagenase.

5. (canceled)

6. The method of claim 3, wherein the enzyme cocktail further comprises DNAse.

7. (canceled)

8. (canceled)

9. (canceled)

10. (canceled)

11. (canceled)

12. (cancel)

13. The method of claim 1, wherein the MSCs are cultured to at least 85% confluency prior to pre-activating and/or wherein the MSCs are cultured for 6 to 8 days prior to pre-activating.

14. (canceled)

15. (canceled)

16. The method of claim 1, wherein the pre-activating is for 12 to 24 hours.

17. (canceled)

18. The method of claim 1, wherein the MSCs are pre-activated in the presence of TNFα, IFNγ, IL-1β, and IL-17.

19. (canceled)

20. (canceled)

21. (canceled)

22. (canceled)

23. (canceled)

24. (canceled)

25. (canceled)

26. The method of claim 1, wherein expanding is performed for less than 7 days.

27. (canceled)

28. (canceled)

29. (canceled)

30. (canceled)

31. The method of claim 1, wherein the population of expanded MSCs has a higher immunosuppressive phenotype as compared to bone marrow MSCs or wherein the population of expanded MSCs has a higher immunosuppressive phenotype as compared to cord tissue-derived MSCs expanded without cytokine pre-activation.

32. (canceled)

33. (canceled)

34. (canceled)

35. (canceled)

36. (canceled)

37. (canceled)

38. The method of claim 1, wherein the population of expanded MSCs has increased expression of stemness markers and/or chemokine receptors as compared to bone marrow-derived MSCs.

39. (canceled)

40. (canceled)

41. The method of claim 1, wherein the population of expanded MSCs has increased expression of genes related to adhesion and invasion as compared to bone marrow-derived MSCs.

42. (canceled)

43. (canceled)

44. (canceled)

45. A composition for the dissociation of cord tissue comprising collagenase, hyaluronidase, and DNase.

46. (canceled)

47. The method of claim 45, wherein the composition consists of collagenase, hyaluronidase, and DNase.

48. The method of claim 45, wherein the composition does not comprise BSA or a trypsin inhibitor.

49. (canceled)

50. (canceled)

51. (canceled)

52. (canceled)

53. (canceled)

54. (canceled)

55. (canceled)

56. A pharmaceutical composition comprising the expanded MSCs produced by the method of claim 1, and a pharmaceutically acceptable carrier.

57. (canceled)

58. A method of treating an inflammatory disease in a subject comprising administering to said subject a therapeutically effective amount of the cord-tissue derived MSCs produced by the method of claim 1.

59. (canceled)

60. (canceled)

61. The method of claim 58, wherein the cord-tissue derived MSCs have been previously cryopreserved.

62. The method of claim 58, wherein the inflammatory disease is graft versus host disease (GVHD), an autoimmune disease, acute ischemic stroke, myocardial damage, acute respiratory distress syndrome (ARDS), or inflammatory bowel disease.

63. (canceled)

64. (canceled)

65. (canceled)

66. The method of claim 58, wherein the MSCs are administered in conjunction with at least one additional therapeutic agent.

67. (canceled)

68. (canceled)

69. (canceled)

70. (canceled)
